(19) United States
(12) Patent Application Publication
(10) Pub. No.: US 2022/0074949 A1
(43) Pub. Date: Mar. 10, 2022

(54) METHODS OF PREPARING SAMPLES FOR PROTEOMIC ANALYSIS

(71) Applicant: STRECK, INC., La Vista, NE (US)

(72) Inventor: Matthew R. Sobansky, La Vista, NE (US)

(21) Appl. No.: 17/417,234

(22) PCT Filed: Dec. 27, 2019
(86) PCT No.: PCT/US2019/068743

§ 371 Date: Jun. 22, 2021

Related U.S. Application Data
(60) Provisional application No. 62/785,501, filed on Dec. 27, 2018.

(51) Int. Cl. G01N 33/68; G01N 1/40; G01N 30/72; G01N 30/88; G01N 30/86

(52) U.S. Cl. CPC G01N 33/6848; G01N 1/4044; G01N 30/7233; G01N 2030/027; G01N 30/8624; G01N 2030/8831; G01N 30/88

(57) ABSTRACT

Provided herein are methods of preparing a protein sample for proteomic analysis. In exemplary embodiments, the method comprises (a) contacting a blood sample comprising proteins with a protective agent comprising an anticoagulant (AC) and an aldehyde releaser (AR), to obtain a mixture, optionally, wherein the blood sample is added to a blood collection tube (BCT) comprising the protective agent, and (b) isolating a fraction comprising proteins or a source of proteins from the mixture to yield a protein sample or a source of a protein sample, wherein steps of the method are carried out in the absence of exogenous proteolytic enzyme inhibitors, wherein the protein sample is suitable for proteomic analysis. Preferably, the protective agent consists of essentially (i) about 300 g/l to about 700 g/l imidazolidinyl urea; (ii) about 20 g/l to about 60 g/l glycine; and (iii) about 60 g/l to about 100 g/l EDTA; and the protein sample is analysed via mass spectrometry-based proteomic methods.

### CROSS REFERENCE TO RELATED APPLICATIONS

This application claims priority to U.S. Provisional Application No. 62/785,501, filed Dec. 27, 2018, the entire contents of which is incorporated herein by reference.

### BACKGROUND

The term “proteome”, first coined by Marc Wilkins, refers to the entire set of proteins expressed by a cell, tissue or organism. Accordingly, “proteomics” is the study and characterization of the proteome present in a cell, organ, or organism at a given point in time. Proteomes, unlike genomes, are complicated due to temporal and spatial variations. Post-translational modifications which frequently regulate protein activity further make the study of proteomes very complex. These proteome variations and alterations, however, find great utility for identifying molecular signatures which serve as diagnostic tools for a variety of diseases. The field of clinical proteomics is dedicated to the identification and validation of such molecular signatures in the context of disease. Ahmad et al., J Proteomics and Genomics 1(1): 103 (2014). The techniques used by clinical proteomic researchers continue to grow in number, and include methods involving the basic techniques of electrophoresis and immunoassay, to more complicated methods like mass spectrometry, or a version thereof, including, for instance, electron-spray ionization (ESI), liquid chromatography coupled mass spectrometry (LC-MS), and surface enhanced laser desorption ionization (SELDI). The field of quantitative proteomics has emerged for the absolute quantification of proteins in a given sample and includes techniques, such as isotopic coded affinity tags (ICAT), stable isotopic labeling with am (SILAC) and isobaric tags for relative and absolute quantification (iTRAQ). Ahmad et al., 2014, supra.

Regardless of the techniques utilized, of core importance to the quality, accuracy and reliability of the data produced by the proteomic analysis is the stability of the sample. “Preanalytical techniques especially sample storage, transportation and processing are the key factors for effective and unbiased results. Loss of less abundant proteins, or protein modifications during repeated freeze thaw cycles, or improper storage are known to affect the results.” Ahmad et al., 2014, supra. Samples are generally unstable at room temperature and must be stored at −80° C. In addition to avoiding repeated freeze thaw cycles, care must be taken to avoid proteases, and the addition of commercially available protease inhibitors to the sample during or after preparing samples from raw blood or tissue is the traditional course of action to address that issue. “In conclusion, one can say that they biggest issues with sample storage and stability in proteomics are temperature and environmental proteases.” Ahmad et al., 2014, supra.

Often times, storing samples, e.g., blood samples, at refrigerated temperatures soon after collection is not possible or inconvenient. Additionally, commercially available protease inhibitors can be costly when a multiplicity of samples is involved in the proteomic analysis. Thus, there is a need in the art for improved methods of preparing samples for proteomic analysis.

### SUMMARY

Presented herein for the first time are data demonstrating the feasibility of collecting whole blood in blood collection tubes (BCTs) comprising a protective agent, as described herein, and subsequently processing the collected blood samples for proteomic analysis, in the absence of additional exogenous proteolytic enzyme inhibitors. Without being bound to any particular theory, the use of the protective agent, as described herein, provides a number of benefits to the preparation of protein samples for proteomic analysis. As further described herein, the protective agent stabilizes cells (e.g., red blood cells, white blood cells, platelets), thereby reducing or preventing unwanted cell lysis and the subsequent release of cellular proteins into the sample. In certain instances, such cellular proteins are considered contaminants to the protein sample for proteomic analysis, because these cellular proteins can mask the presence of low-abundance proteins in the sample and impede the identification and/or quantification of proteins in the sample. Without being bound to any particular theory, the protective agent also inactivates proteolytic enzymes in the sample and thus decreases or eliminates the requirement for additional proteolytic enzyme inhibitors. The use of the protective agent in the presently disclosed methods reduces the impact of proteolytic enzyme-mediated degradation and increases the stability of the collected blood samples.

Accordingly, the present disclosure provides a method of preparing a protein sample for proteomic analysis. In exemplary embodiments, the method comprises (a) contacting a blood sample comprising proteins with a protective agent comprising an anticoagulant (AC) and an aldehyde releaser (AR), to obtain a mixture, optionally, wherein the blood sample is added to a blood collection tube (BCT) comprising the protective agent or the blood sample is directly drawn from a subject into a BCT comprising the protective agent, and (b) isolating a fraction comprising proteins from the mixture to yield a protein sample suitable for proteomic analysis. In exemplary aspects, steps (a) and (b) of the method are carried out in the absence of additional exogenous proteolytic enzyme inhibitors (e.g., steps (a) and (b) of the method are carried out without the addition or use of exogenous proteolytic enzyme inhibitors outside of the protective agent). In exemplary aspects, the slope of the best fit line of a line graph of the number of proteins in the protein sample yielded from step (b) plotted as a function of storage time is closer to 0 compared to the slope of the best fit line of a line graph of the number of proteins in a control blood sample not contacted with a protective agent. In exemplary aspects, the number of plasma proteins and/or peptides present in the protein sample following storage for at least 48 hours is within about 10% of the number of plasma proteins and/or peptides present in the protein sample within about 0 hours to about 4 hours of collecting the blood sample from a subject. In exemplary aspects, the method further comprises transporting the mixture in a sealed container to a laboratory for proteomic analysis, optionally, wherein the sealed container is a sealed BCT comprising the protective agent. In exemplary embodiments, the method comprises (a) contacting a blood sample comprising proteins with a protective agent comprising an AC and an AR, to obtain a mixture, optionally, wherein the blood sample is added to a BCT comprising the protective agent or the blood sample is directly drawn from a subject into a BCT comprising the protective agent, (b) isolating a cellular fraction comprising a source of cellular proteins from the mixture, and (c) optionally lysing cells of the cellular fraction to yield a protein sample comprising cellular proteins. In exemplary aspects, the protein sample is suitable for proteomic analysis, and wherein steps (a) and (b) of the method are carried out in the absence of additional exogenous proteolytic enzyme inhibitors (e.g., steps (a) and (b) of the method are carried out without the addition or use of exogenous proteolytic enzyme inhibitors outside of the protective agent). In exemplary aspects, the slope of the best fit line of a line graph of the number of proteins in the protein sample yielded from step (b) plotted as a function of storage time is closer to 0 compared to the slope of the best fit line of a line graph of the number of proteins in a control blood sample not contacted with a protective agent. In exemplary aspects, the number of plasma proteins and/or peptides present in the protein sample following storage for at least 48 hours is within about 10% of the number of plasma proteins and/or peptides present in the protein sample within about 0 hours to about 4 hours of collecting the blood sample from a subject. In exemplary aspects, the method further comprises transporting the mixture in a sealed container to a laboratory for proteomic analysis, optionally, wherein the sealed container is a sealed BCT comprising the protective agent. As further described herein, in some aspects, the protective agent comprises an AC and an AR, wherein the AC is functions as both an AC and a proteolytic enzyme inhibitor, and the method lacks the addition or use of any exogenous proteolytic enzyme inhibitors, outside of the protective agent. In exemplary embodiments, the method comprises (a) adding a blood sample comprising proteins into a BCT comprising a protective agent consisting essentially of (i) about 300 g/l to about 700 g/l imidazolidinyl urea; (ii) about 20 g/l to about 60 g/l glycine; and (iii) about 50 g/l or about 60 g/l to about 100 g/l EDTA to obtain a mixture; (b) optionally, storing the blood sample in the BCT for at least about 48 hours at about 20° C. to about 25° C., (c) isolating a fraction comprising proteins, yielding a protein sample suitable for proteomic analysis and (d) analyzing the protein sample via one or more mass spectrometry-based proteomic methods. In exemplary embodiments, the method comprises (a) adding a blood sample comprising proteins into a BCT comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA to obtain a mixture; (b) optionally, storing the mixture for at least about 48 hours at about 20° C. to about 25° C., (c) isolating a cellular fraction comprising a source of cellular proteins from the mixture, (d) lysing cells of the cellular fraction to yield a protein sample comprising cellular proteins, wherein the protein sample is suitable for proteomic analysis and (e) analyzing the protein sample via one or more mass spectrometry-based proteomic methods. In exemplary aspects, adding a blood sample comprising proteins into a BCT comprising a protective agent comprises directly drawing the blood sample from a subject in the BCT comprising the protective agent.

### BRIEF DESCRIPTION OF THE DRAWINGS

FIG. 1A provides an overlay total ion chromatogram (TIC) plot of the Tube 1 whole plasma sample (red trace) and the Tube 2 whole plasma sample (green trace).

FIG. 1B and FIG. 1C are views of the Tube 2 whole plasma sample (FIG. 1B) and the Tube 1 whole plasma sample (FIG. 1C).

FIG. 2A provides an overlay TIC plot of the Tube 1 depleted plasma sample (red trace) and the Tube 2 depleted sample (green trace).

FIGS. 2B and 2C are stacked view of the Tube 2 depleted sample (FIG. 2B) and the Tube 1 depleted sample (FIG. 2C).

FIG. 3A is a graph of the number of proteins in the sample obtained from Donor A collected the CF BCT (Tube 1) or the E BCT (EDTA) plotted as a function of storage time.

FIG. 3B is a graph of the number of proteins in the sample obtained from Donor B collected the CF BCT (Tube 1) or the E BCT (EDTA) plotted as a function of storage time. FIG. 3C is a graph of the number of proteins in the sample obtained from Donor C collected the CF BCT (Tube 1) or the E BCT (EDTA) plotted as a function of storage time. For each figure, the best fit lines are shown as dotted lines.

FIG. 4A is a graph of the number of peptides in the sample obtained from Donor A collected the CF BCT (Tube 1) or the E BCT (EDTA) plotted as a function of storage time.

FIG. 4B is a graph of the number of peptides in the sample obtained from Donor B collected the CF BCT (Tube 1) or the E BCT (EDTA) plotted as a function of storage time. FIG. 4C is a graph of the number of peptides in the sample obtained from Donor C collected the CF BCT (Tube 1) or the E BCT (EDTA) plotted as a function of storage time. For each figure, the best fit lines are shown as dotted lines.

FIG. 5 is a dendogram obtained from the hierarchal clustering analyses.

Each of FIGS. 6A-6E is a graph of protein concentration plotted as a function of time for each sample collected in a CF BCT (Tube 1) or in E BCTs (EDTA) from Donors A-C. Transketolase (FIG. 6A); Rho GDP dissociation inhibitor 2 (FIG. 6B); Phosphoglycerate Kinase 1 (FIG. 6C), Profilin (FIG. 6D); Hemoglobin Subunit Delta (FIG. 6E).

FIG. 7 is a series of example chromatograms of samples stored for 0 hrs to 216 hours for samples collected in a CF BCT (Tube 1) or in E BCTs (EDTA) from Donors A-C. The lines are the extracted ion chromatograms for peptide ions used to quantify levels of Profilin-1 (P07737) in samples stored in BCTs over time. Increased peak intensity (height) corresponds to increased Profilin-1 levels. These data demonstrate that samples collected in Tube 1 had delayed degradation (no increase in intensity at 48 hours) compared to E tubes.

Each of FIGS. 8A-8D of protein concentration plotted as a function of time for each sample collected in a CF BCT (Tube 1) or in E BCTs (EDTA) from Donors A-C. Platelet Factor 4 (FIG. 8A); Platelet basic protein (FIG. 8B); von Willebrand factor (FIG. 8C); Fibronectin (FIG. 8D). The inset graphs show 0-24 hours at increased detail to demonstrate that levels are equivalent at T=0 for both tubes. Insert also shows that Tube 1 reaches rapid equilibrium while EDTA changes slowly over time.

FIG. 9 is a series of example chromatograms of samples stored for 0 hrs to 216 hours for samples collected in a CF BCT (Tube 1) or in E BCTs (EDTA) from Donors A-C. The lines are the extracted ion chromatograms for peptide ions used to quantify levels of Platelet basic protein in samples stored in BCTs over time. Increased peak intensity (height) corresponds to increased Platelet basic protein levels. These data demonstrate that samples collected in Tube 1 had delayed degradation (no increase in intensity at 48 hours) compared to E tubes.

FIG. 10 is an SDS-PAGE gel of undepleted plasma (PL), or depleted plasma samples (T12 and T2) which were depleted using the Top 12 or Top 2 protein depletion techniques described herein.

FIG. 11 is a graph of the average number of quantifiable proteins of samples collected in CF BCTs (Tube 1) or with E tubes (EDTA), plotted as a function of time.

FIGS. 12A-12E are graphs of the amount of the indicated protein over the course of time in samples collected in CF BCTs from Donors A-C (A-Tube 1, B-Tube, C-Tube 1), or collected in E tubes from Donors A-C (A-EDTA, B-EDTA, C-EDTA).

FIGS. 13A-13B are graphs of the amount of the indicated protein over the course of time in samples collected in CF BCTs from Donors A-C (A-Tube 1, B-Tube, C-Tube 1), or collected in E tubes from Donors A-C (A-EDTA, B-EDTA, C-EDTA).

### DETAILED DESCRIPTION

Provided herein are methods of preparing a protein sample for proteomic analysis wherein blood cell lysis and protease activity within the blood sample are reduced. Without being bound to any particular theory, the protective agent stabilizes cells and reduces the degradation of proteins which are the analytes of the proteomic analysis, such that the blood sample comprising proteins may be stored for longer periods of time at temperatures higher than refrigerated temperatures.

The present disclosure provides a method of preparing a protein sample for proteomic analysis. In exemplary embodiments, the method comprises (a) contacting a blood sample comprising proteins with a protective agent comprising an AC and an AR to obtain a mixture. In exemplary aspects, the blood sample is contacted with the protective agent by adding the blood sample comprising proteins to a BCT comprising the protective agent. In exemplary aspects, the blood sample is directly drawn from a subject into a BCT comprising the protective agent. In alternative aspects, the blood sample is contacted with the protective agent by adding the protective agent to the blood sample. In exemplary embodiments, the protective agent comprises an AC which functions as both an AC and a proteolytic enzyme inhibitor and the method does not comprise the use of any additional exogenous proteolytic enzyme inhibitors (outside the protective agent). In exemplary embodiments, the method further comprises (b) isolating a fraction comprising proteins from the mixture, thereby yielding a protein sample suitable for proteomic analysis. In exemplary embodiments, the method further comprises (b) isolating a cellular fraction comprising a source of cellular proteins from the mixture, and (c) optionally lysing cells of the cellular fraction to yield a protein sample comprising cellular proteins, wherein the protein sample is suitable for proteomic analysis.

In exemplary embodiments, the method comprises (a) adding a blood sample comprising proteins into a BCT comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA to obtain a mixture; (b) optionally, storing the blood sample in the BCT for at least about 48 hours at about 20° C. to about 25° C., (c) isolating a fraction comprising proteins, yielding a protein sample suitable for proteomic analysis and (d) analyzing the protein sample via one or more mass spectrometry-based proteomic methods. In exemplary embodiments, the method comprises (a) adding a blood sample comprising proteins into a BCT comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA to obtain a mixture; (b) optionally, storing the mixture for at least about 48 hours at about 20° C. to about 25° C., (c) isolating a cellular fraction comprising a source of cellular proteins from the mixture, (d) lysing cells of the cellular fraction to yield a protein sample comprising cellular proteins, wherein the protein sample is suitable for proteomic analysis and (d) analyzing the protein sample via one or more mass spectrometry-based proteomic methods. In exemplary aspects, adding a blood sample comprising proteins into a BCT comprising a protective agent comprises directly drawing the blood sample from a subject in the BCT comprising the protective agent.

Protective Agent

As used herein, the term “protective agent” refers to a composition comprising components which function together to (i) preserve cell morphology, stabilize cell structure, and/or prevent or reduce cell degradation, thereby reducing or preventing cell lysis and subsequent release of cellular proteins, and (ii) prevent or reduce protein degradation through the actions of deleterious proteolytic enzymes (e.g., thrombin, plasmin). The protective agent allows for stabilization of the blood sample. In some aspects, the protective agent is a solid, liquid, gel, or other semi-solid. In some aspects, the protective agent is a liquid. In exemplary aspects, the protective agent comprises an anticoagulant (AC) and an aldehyde releaser (AR), optionally with one or more additional components, as described herein. In some aspects, the protective agent comprising an AC and AR is in solution. Suitable solvents include water, saline, dimethylsulfoxide, alcohol or a mixture thereof. The protective agent may comprise additional components, e.g., diazolidinyl urea (DU) and/or imidazolidinyl urea (IDU), optionally in a buffered salt solution. The protective agent may be present in a BCT as a liquid in an amount less than about 10% by volume of the BCT but greater than about 0.1% by volume. The protective agent may be present in the BCT as a liquid in an amount less than about 5% by volume of the BCT but greater than about 0.1% by volume of the BCT. The protective agent may be present in the BCT in an amount less than about 3% by volume of the BCT but greater than about 0.1% by volume of the BCT.

In exemplary embodiments, the protective agent is substantially non-toxic and/or chemically inert with respect to the blood sample and any components thereof, e.g., cells, proteins, nucleic acids, exosomes, and the like. In various aspects, the protective agent is substantially free of formaldehyde, paraformaldehyde, guanidinium salts, sodium dodecyl sulfate (SDS), or any combination thereof. In some aspects, the protective agent is substantially free of formaldehyde. For instance, the protective agent comprises formaldehyde in an amount that is less than or about 50,000 ppm. The protective agent in some aspects comprises less than about 20 parts per million (ppm) of formaldehyde. The protective agent may contain less than about 15 parts per million (ppm) of formaldehyde. The protective agent may contain less than about 10 parts per million (ppm) of formaldehyde. The protective agent may contain less than about 5 parts per million (ppm) of formaldehyde. The protective agent may contain at least about 0.1 parts per million (ppm) to about 20 ppm of formaldehyde. The protective agent may contain at least about 0.5 parts per million (ppm) to about 15 ppm of formaldehyde. The protective agent may contain at least about 1 parts per million (ppm) to about 10 ppm of formaldehyde.

As further described herein, the protective agent is substantially free of proteolytic enzyme inhibitors which do not also function as an AC. As used herein, the term “proteolytic enzyme inhibitors” refer to any agent, chemical (e.g., small molecule) or biological, naturally occurring or synthetic, which inhibits a protease or proteinase. Proteases are classified by their mechanism of action, and include for example, serine proteases, cysteine (thiol) proteases, aspartic proteases, metalloproteases, endoproteases, trypsin-like proteases, chymotrypsin-like proteases, caspase-like proteases, elastase-like proteases. In various aspects, the proteolytic enzyme inhibitor reduces the activity of one or more of these proteases. Proteolytic enzyme inhibitors are known in the art and include, but are not limited to: alpha-2-macroglobulin, 4-(2-Aminoethyl)benzenesulfonyl fluoride (AEBSF), Amidinophenylmethanesulfonyl fluoride hydrochloride; (APMSF), amastatin, antipain, aprotinin, bestatin, chymostatin, diprotin A, diprotin B, EDTA, E-64, egg white cystatin, egg white ovostatin, elastatinal, galardin, indoleacetic acid (IAA), leupeptin, trypsin inhibitors (e.g., soybean trypsin inhibitor), nelfinavir mesylate, pepstatin (e.g., pepstatin A), phenylmethylsulfonyl fluoride (PMSF), phosphoramodon, 1,10-phenanthroline, pancreatic protease inhibitor, 4-Tosyl-L-lysyl-chloromethane hydrochloride (TLCK), Tosyl phenylalanyl chloromethyl ketone (TPCK), VdLP FFVdL, and any combination thereof. The protective agent is substantially free of these inhibitors or any combination thereof, including any combination of proteolytic enzyme inhibitors sold by commercial suppliers and referred to as a “protease inhibition cocktails”. Mixtures, combinations, or cocktails of protease inhibitors are also known in the art, including Roche cOmplete tablets, Roche cOmplete ULTRA (EDTA-free) protease inhibitor cocktail tablet, Calbiochem protease inhibitor cocktail, Halt Protease Inhibitor Cocktail, G-Biosciences FOCUS™ ProteaseArrest™, Recom ProteaseArrest™.

Proteolytic enzyme inhibitors which also function as an AC include, but are not limited to, EDTA and EGTA. In exemplary aspects, such components are be used as an AC of the protective agent but are not used outside of the protective agent, e.g., the method does not comprise a step of using additional EDTA or EGTA outside of the EDTA or EGTA already present in the protective agent.

In exemplary aspects, the protective agent comprises imidazolidinyl urea, EDTA, and glycine, and is substantially free of any proteolytic enzyme inhibitors which do not also function as an AC. In exemplary instances, the protective agent comprises imidazolidinyl urea at a concentration of about 300 g/l to about 700 g/l. In exemplary instances, the protective agent comprises EDTA at a concentration of about 60 g/l to about 100 g/l EDTA. In exemplary instances, the protective agent comprises glycine at a concentration of about 20 g/l to about 60 g/l glycine. In various instances, the protective agent consists essentially of (i) about 300 g/l to about 700 g/l imidazolidinyl urea; (ii) about 20 g/l to about 60 g/l glycine; and (iii) about 60 g/l to about 100 g/l EDTA.

Anticoagulant

The protective agent of the presently disclosed BCTs comprises an anticoagulant (AC) (i.e., an agent that inhibits the coagulation of blood). In exemplary aspects, the AC is ethylene diamine tetra acetic acid (EDTA) or a salt thereof, ethylene glycol tetra acetic acid (EGTA) or a salt thereof, hirudin, heparin, citric acid, a salt of citric acid, oxalic acid, a salt of oxalic acid, acid citrate dextrose (ACD; also known as anticoagulant citrate dextrose), citrate-theophylline-adenosine-dipuridamole (CTAD), citrate-pyridoxalphosphate-tris, heparin-1,3-hydroxy-ethyl-theophylline, polyanethol sulfonate, sodium polyanethol sulfonate, sodium fluoride, sodium heparin, thrombin and PPACK (D-phenylalanyl-L-prolyl-L-arginine chloromethyl ketone), or a combination thereof. In exemplary aspects, the anticoagulant is EDTA. Optionally, EDTA is the only AC present in the protective agent.

In exemplary aspects, the AC is present in the protective agent in an amount of about 10 g/L to about 500 g/L, about 10 g/L to about 450 g/L, about 10 g/L to about 400 g/L, about 10 g/L to about 350 g/L, about 10 g/L to about 300 g/L, about 10 g/L to about 250 g/L, about 10 g/L to about 200 g/L, about 10 g/L to about 150 g/L, about 10 g/L to about 100 g/L, about 10 g/L to about 75 g/L, about 10 g/L to about 50 g/L, about 50 g/L to about 500 g/L, about 75 g/L to about 500 g/L, about 100 g/L to about 500 g/L, about 150 g/L to about 500 g/L, about 200 g/L to about 500 g/L, about 250 g/L to about 500 g/L, about 300 g/L to about 500 g/L, about 350 g/L to about 500 g/L, about 400 g/L to about 500 g/L, about 450 g/L to about 500 g/L, about 20 g/L to about 400 g/L, about 30 g/L to about 300 g/L, about 40 g/L to about 250 g/L, or about 50 g/L to about 200 g/L. In certain instances, the anticoagulant is present in the protective agent in an amount of about 50 g/L to about 150 g/L or about 60 g/L to about 100 g/L. In some aspects, about 50 g/L, about 60 g/L, about 70 g/L, about 80 g/L, about 90 g/L, or about 100 g/L anticoagulant is present in the protective agent. In exemplary instances, the protective agent comprises EDTA at a concentration of about 60 g/l to about 100 g/l EDTA. In exemplary aspects, the protective agent comprises about 30 g/L to about 100 g/L anticoagulant, optionally, about 30 g/L to about 100 g/L anticoagulant, about 40 g/L to about 100 g/L anticoagulant, about 50 g/L to about 100 g/L anticoagulant, about 60 g/L to about 100 g/L anticoagulant, about 70 g/L to about 100 g/L anticoagulant, about 80 g/L to about 100 g/L anticoagulant, about 90 g/L to about 100 g/L anticoagulant, about 30 g/L to about 90 g/L anticoagulant, about 30 g/L to about 80 g/L anticoagulant, about 30 g/L to about 70 g/L anticoagulant, about 30 g/L to about 60 g/L anticoagulant, about 30 g/L to about 50 g/L anticoagulant, or about 30 g/L to about 40 g/L anticoagulant.

Aldehyde Releaser

The protective agent comprises an aldehyde releaser (AR) (i.e., an agent that reacts to form an aldehyde product, e.g., a formaldehyde product). In exemplary aspects, the AR reacts to provide a slow release of the aldehyde product over time and without being bound to a particular theory, the slow release of the aldehyde product by the AR imparts stability to the blood sample, e.g., the cellular components of the blood sample. In exemplary embodiments, the aldehyde releaser is diazolidinyl urea, imidazolidinyl urea, 1,3,5-tris(hydroxyethyl)-s-triazine, oxazolidine, 1,3-bis(hydroxymethyl)-5,5-dimethylimidazolidine-2,4-dione, quaternium-15, DMDM hydantoin, 2-bromo-2-nitropropane-1,3-diol, 5-bromo-5-nitro-1,3-dioxane, tris(hydroxymethyl) nitromethane, hydroxymethylglycinate, polyquaternium, or a combination thereof. In exemplary aspects, the aldehyde releaser is imidazolidinyl urea. Optionally, imidazolidinyl urea is the only AR in the protective agent.

In exemplary aspects, the aldehyde releaser is present in the protective agent in an amount of about 100 g/L to about 1000 g/L, about 200 g/L to about 1000 g/L, or about 300 g/l to about 1000 g/l, about 500 g/L to about 1000 g/L, about 600 g/L to about 1000 g/L, about 700 g/L to about 1000 g/L, about 800 g/L to about 1000 g/L, about 900 g/L to about 1000 g/L, about 100 g/L to about 900 g/L, about 100 g/L to about 800 g/L, about 100 g/L to about 700 g/L, about 100 g/L to about 600 g/L, about 100 g/L to about 500 g/L, about 100 g/L to about 400 g/L, about 100 g/L to about 300 g/L, about 100 g/L to about 200 g/L, (e.g., about 300 g/l, about 400 g/l, about 500 g/l, about 600 g/l, or about 700 g/l). In exemplary aspects, the aldehyde releaser is present in the protective agent in an amount of about 100 g/L, about 200 g/L, about 300 g/L, about 400 g/L, about 500 g/L, about 600 g/L, about 700 g/L, about 800 g/L, about 900 g/L, or about 1000 g/L, ±10% g/L. In exemplary instances, the protective agent comprises imidazolidinyl urea at a concentration of about 300 g/l to about 700 g/l.

In exemplary aspects, the protective agent comprises the AR and the AC at a AR to AC ratio of about 1:2 to about 1:6. In some aspects, the protective agent comprises the AR and the AC at a AR to AC ratio of about 1:3 to about 1:5. Optionally, the protective agent comprises the AR and the AC at a AR to AC ratio of about 1:4.

Additional Components of the Protective Agent

The protective agent in some aspects comprises components in addition to the anticoagulant and aldehyde releaser. In exemplary aspects, the protective agent further comprises an amine. The amine in some aspects is a primary amine or secondary amine. In some aspects, the amine is a tertiary amine. In various instances, the amine is an alkylamine, an arylamine, or an alkylarylamine. In exemplary aspects, the amine is an amino acid, biogenic amine, trimethylamine, or aniline. In some aspects, the amino acid is tryptophan, tyrosine, phenylalanine, glycine, ornithine and S-adenosylmethionine, aspartate, glutamine, alanine, arginine, cysteine, glutamic acid, glutamine, histidine, leucine, lysine, proline, serine, threonine, or a combination thereof. In exemplary aspects, the protective agent comprises glycine. Optionally, the glycine is the only amine present in the proteactive agent.

In exemplary aspects, the protective agent comprises about 20 g/l to about 60 g/l amine, about 20 g/L to about 50 g/L, about 20 g/L to about 40 g/L, about 20 g/L to about 30 g/L, about 20 g/L to about 25 g/L, about 25 g/L to about 50 g/L, about 30 g/L to about 50 g/L, or about 40 g/L to about 50 g/L. In exemplary aspects, the amount of aldehyde releaser relative to an amount of amine is about 10 parts by weight of aldehyde releaser to about 1 part by weight amine. Optionally, the amount of aldehyde releaser to the amount of amine is about 15 parts by weight to about 1 part by weight or about 20 parts by weight to about 1 part by weight. In various aspects, the amount of aldehyde releaser to the amount of amine is about 7.5 parts by weight to about 1 part by weight or about 5 parts by weight to about 1 part by weight. In exemplary aspects, the BCT comprises imidazolidinyl urea (IDU) and glycine at a ratio of imidazolidinyl urea (IDU) to glycine may be about 10:1.

In exemplary aspects, the protective agent further comprises one or more preservative agents, enzyme inhibitors, metabolic inhibitors, or a combination thereof. The one or more enzyme inhibitors in some aspects is diethyl pyrocarbonate, ethanol, aurintricarboxylic acid (ATA), glyceraldehydes, sodium fluoride, formamide, vanadyl-ribonucleoside complexes, macaloid, heparin, hydroxylamine-oxygen-cupric ion, bentonite, ammonium sulfate, dithiothreitol (DTT), beta-mercaptoethanol, cysteine, dithioerythritol, tris (2-carboxyethyl) phosphene hydrochloride, a divalent cation (such as Mg⁺², Mn⁺², Zn⁺², Fe⁺², Ca⁺², Cu⁺²) or any combination thereof. In exemplary instances, the protective agent comprises one or more nuclease inhibitors, e.g., DNAse inhibitor or RNase inhibitor. The one or more metabolic inhibitors in certain aspects is glyceraldehyde, dihydroxyacetone phosphate, glyceraldehyde 3-phosphate, 1,3-bisphosphoglycerate, 3-phosphoglycerate, 2-phosphoglycerate, phosphoenolpyruvate, pyruvate or glycerate dihydroxyacetate, sodium fluoride, K₂C₂O₄ or a combination thereof. In exemplary aspects, the protective agent does not comprise a preservative agent, enzyme inhibitor, metabolic inhibitor, described above.

Blood Collection Tubes (BCTs)

The protective agent in exemplary aspects is added to the blood sample. In other aspects, the protective agent is present in a BCT and the blood sample is added to the BCT comprising the protective agent. Suitable BCTs are known in the art and include those described in International Patent Publication No. WO2018145005 and U.S. Pat. No. 9,657,227. The BCTs used in the presently disclosed methods may be made of any suitable, non-toxic, chemically-inert material, such as a plastic, glass, silica, carbon, or a combination thereof. In exemplary aspects, the BCT is made of a material which minimizes adhesion or adherence of cells or proteins or other components of the blood sample. In some aspects, the tube is made of a transparent material. In various instances, the tube is composed of a material comprising polypropylene, polystyrene, or glass (e.g., borosilicate glass, flint glass, aluminosilicate glass, soda lime glass, lead or quartz glass). In exemplary instances, the tube is composed of a material comprising a cyclic polyolefin, e.g., a cyclic polyolefin copolymer or cyclic polyolefin polymer. In exemplary aspects, the materials may be stable at a temperature of about −100° C. to about 50° C. (e.g., 2° C. to about 30° C.) and thus may be suitable for storing samples in a freezer, refrigerator, heater, heated incubator, heated water bath, or at room temperature.

The BCTs may have any geometry or suitable shape for containing and storing a liquid. In exemplary aspects, the tube is substantially cylindrical in shape with one closed end and one open end. In exemplary aspects, the tube comprises an enclosed base, a coextensive elongated side wall extending from the base and terminating at an open end, such that a hollow chamber having an inner wall is defined. In certain aspects, the hollow chamber is configured for collecting a blood sample. In various aspects, at least the elongated side wall of the tube is made of a material including a thermoplastic polymeric material having a high moisture barrier and low moisture absorption rate, and optical transparency to enable viewing a sample within the tube and chemical resistance. The closed end or enclosed base in some aspects is round-bottomed or U-shaped, conical or V-shaped. The open end in various instances comprises a series of threads suitable for fitting a screw cap for temporary closure. In alternative aspects, the open end does not comprise a series of threads. In various instances, the open end may be fitted with a stopper or a cap. In exemplary aspects, the closed end or the enclosed base of the tube is flat. In various instances, at least a portion of the elongated side wall of the tube tapers to a point located at or within the enclosed base or closed end. The BCT in some aspects has an outer diameter, as measured at the coextensive elongated side wall adjacent the open end, to length (D×L) dimension of about 13 mm×75 mm. The BCT in some aspects has an outer diameter, as measured at the coextensive elongated side wall adjacent the open end, to length (D×L) dimension of about 16 mm×100 mm. In alternative instances, the elongated side wall of the tube does not taper to a point.

In various aspects, the volumetric capacity of the tube is at least or about 0.5 mL, at least or about 1 mL, at least or about 1.5 mL, at least or about 2 mL, at least or about 2.5 mL, at least or about 3 mL, at least or about 3.5 mL, at least or about 4 mL, at least or about 4.5 mL, or at least or about 5 mL, and optionally up to about 350 mL, up to about 300 mL, up to about 250 mL, up to about 200 mL, up to about 150 mL, or up to about 100 mL. In some instances, the tube can hold about 1 mL to about 45 mL, about 1 mL to about 40 mL, about 1 mL to about 35 mL, about 1 mL to about 30 mL, about 1 mL to about 25 mL, about 1 mL to about 20 mL, about 1 mL to about 15 mL, about 1 mL to about 10 mL. In various aspects, the volumetric capacity is about 5 mL to about 40 mL, about 5 mL to about 30 mL, about 5 mL to about 20 mL, about 5 mL to about 15 mL, optionally, about 5 mL, about 6 mL, about 7 mL, about 8 mL, about 9 mL, about 10 mL, about 11 mL, about 12 mL, about 13 mL, about 15 mL, about 16 mL, about 17 mL, about 18 mL, about 19 mL, about 20 mL.

The BCT in some aspects includes a reagent fill tolerance volume of about 54 μl to about 66 μl. The BCT in certain instances includes a reagent fill tolerance volume of about 60 μl. The BCT in various aspects comprises a reagent fill tolerance volume of about 162 μl to about 198 μl, optionally, about 180 μl. The BCT may include a reagent fill by weight of plus or minus 10% of 0.0708 g. The BCT may include a reagent fill by weight of plus or minus 10% of 0.224 g. The BCT in some aspects includes a draw tolerance of about 3 ml to about 5 ml. The tube may include a draw tolerance of about 4 ml. The BCT in certain instances includes a draw tolerance of about 7 ml to about 13 ml. The BCT includes in some aspects a draw tolerance of about 9 ml. In exemplary aspects, the BCT has a reagent volume of about 50 μL to about 500 μL, e.g., about 50 μL to about 400 μL, about 50 μL to about 300 μL, about 50 μL to about 200 μL, about 50 μL to about 100 μL, about 100 μL to about 500 μL, about 200 μL to about 500 μL, about 300 μL to about 500 μL, about 400 μL to about 500 μL. In various instances, the BCT has a reagent volume of about 100 μL to about 300 μL or about 150 μL to about 250 μL or about 175 μL to about 225, e.g., about 200 μL. In various aspects, the BCT has a fill volume of about 1 mL to about 100 mL, about 1 mL to about 75 mL, about 1 mL to about 50 mL, about 1 mL to about 25 mL, about 1 mL to about 15 mL, about 1 mL to about 10 mL, about 10 mL to about 100 mL, about 15 mL to about 100 mL, about 25 mL to about 100 mL, about 50 mL to about 100 mL, about 75 mL to about 100 mL. In exemplary aspects, the BCT has a reagent volume of about 200 μL and a fill volume of about 10 mL.

In exemplary instances, the BCT comprises an open end that may be fitted with a cap to at least temporarily seal the end. In exemplary aspects, the BCT comprises threads that function with a screw cap to at least temporarily seal the open end of the tube. In some aspects, the BCT does not comprise any threads. Rather, a stopper or similar cap may be outfitted on the BCT for sealing the open end. The cap may be composed of a non-toxic, chemically-inert material, such as a plastic or rubber. The cap may be a bromobutyl rubber stopper. In some aspects, the stopper of the BCT may include a silicone oil coating over at least a portion of its outer surface that contacts the inner wall of the BCT. The base may include a recessed dimple. In some aspects, the base does not have a dimple.

In exemplary embodiments, the interior wall of the tube is coated or otherwise treated to modify its surface characteristics, such as to render it more hydrophobic and/or more hydrophilic, over all or a portion of its surface. The tube in some aspects has an interior wall flame sprayed, subjected to corona discharge, plasma treated, coated or otherwise treated. In various instances, the tube is treated by contacting an interior wall with a substance so that the proteins of interest do not adhere to the tube walls. The surface of the tube in some aspects are modified to provide multi functionality that simultaneously provides an appropriate balance of desired hydrophilicity and hydrophobicity, to allow collection of blood, dispersion of the preservatives herein, and resistance of adhesion of nucleic acids to the inner wall of a blood collection tube.

The coating, in some aspects, is a silicone coating. In various instances, the coating comprises a functionalized polymer that includes a first polymer and one or more second monomeric and/or polymeric functionalities that are different from (e.g., chemically different from) the first polymer. The coating in some instances include one or more co-polymers (e.g., block copolymer, graft copolymer, or otherwise). For example, it may include a copolymer that includes a first hydrophobic polymeric portion, and a second hydrophilic polymeric portion. The coating may be a water based coating. The coating may optionally include an adhesion promoter. The coating may be applied in any suitable manner, it may be sprayed, dipped, swabbed, or otherwise applied onto some or all of the interior of the blood collection tube. The coating may also be applied in the presence of heat. Preferably any coating applied to the inner wall of a blood collection tube will form a sufficiently tenacious bond with the glass (e.g., borosilicate glass) or other material (e.g., polymeric material) of the tube so that it will not erode or otherwise get removed from the inner wall. Examples of suitable polymeric coatings may include silicon containing polymers (e.g., silanes, siloxanes, or otherwise); polyolefins such as polyethylene or polypropylene; polyethylene terephthalate; fluorinated polymers (e.g., polytetrafluoroethylene); polyvinyl chloride, polystyrene or any combination thereof. Examples of teachings that may be employed to coat an interior of a blood collection tube may be found in U.S. Pat. Nos. 6,551,267; 6,077,235; 5,257,633; and 5,213,765; all incorporated by reference.

Isolating Fractions and Fraction Types

In exemplary embodiments, the presently disclosed methods comprise a step of isolating a fraction comprising proteins. In exemplary aspects, the fraction comprises plasma of the blood sample. In exemplary aspects, the fraction is a plasma fraction. In aspects, the method comprises isolating a plasma fraction from the blood sample to yield a protein sample suitable for proteomic analysis. In various aspects, the plasma fraction is substantially free of cells, e.g., substantially free of red blood cells, white blood cells, platelets.

In various aspects, the fraction is comprises cells of the blood sample. In certain instances, the fraction is a cellular fraction of the blood sample. In exemplary instances, the cellular fraction consists essentially of rare blood cells, optionally, circulating tumor cells (CTCs). Optionally, the cellular fraction is free of red blood cells, white blood cells, platelets, or a combination thereof. The cellular fraction in some instances is free of plasma proteins. In some aspects, the method comprises lysing cells of the cellular fraction to obtain a protein sample suitable for proteomic analysis.

The isolating step of the presently disclosed method in some aspects comprises a centrifugation step. For example, the centrifugation step may be such that the centrifugation step yields a cell pellet and a cell-free supernatant. In exemplary embodiments, the isolating step comprises isolating plasma by, e.g., centrifuging the blood sample at about 2000 g for about 15 minutes. Optionally, the isolated plasma is further centrifuged to obtain clarified plasma. Accordingly, the isolating step may comprise centrifuging the blood sample at about 2000 g for about 15 minutes (optionally at room temperature) to obtain a supernatant comprising isolated plasma, followed by centrifuging the supernatant comprising the isolated plasma at about 16,000 g for about 10 minutes (optionally at room temperature) to obtain a supernatant comprising clarified plasma. In exemplary embodiments, the plasma (e.g., the clarified plasma) may be further processed. For example, the clarified plasma may be depleted of proteins that are present in plasma at a relatively high concentration. In exemplary instances, the clarified plasma is depleted of immunoglobulins, albumin, or a combination of both. Methods of depletion are known in the art and include use of spin trap columns. See, e.g., Example 1.

In alternative or additional embodiments, the isolating step of the presently disclosed methods comprises one or more chromatography steps, electrophoretic separation steps, immunoprecipitation steps, or a combination thereof. Suitable techniques for isolating fractions comprising proteins from blood samples are known in the art.

In some aspects, the isolating step comprises a cell sorting step, e.g., a fluorescence activated cell sorting (FACS) step. The cell sorting step in some aspects is based on expression of a cell surface protein on some cells, or a lack of expression of a cell surface protein on some cells.

In exemplary embodiments, the isolating step yields a protein sample comprising substantially the same amount of proteins (e.g., intact proteins) as in the blood sample upon collection into the BCT. In exemplary embodiments, the isolating step yields a protein sample comprising substantially the same types of proteins as in the blood sample upon collection into the BCT. In other words, the protein sample is substantially the same as the original blood sample in terms of the proteins present in the sample and the amount of each protein. Also, in some embodiments, the protein sample has little to substantially no loss of proteins through protein degradation or protein aggregation. In various aspects, the protein sample has little to substantially no contaminant protein products. In exemplary embodiments, the isolating step yields a protein sample comprising less than about 25% contaminant protein products as measured by high performance liquid chromatography mass spectrometry (HPLC-MS). As used herein, the term “contaminant protein products” refers to unwanted protein products including but not limited to protein fragments, intact intracellular proteins, aggregates of whole proteins and/or protein fragments, and the like which result from degradation, aggregation (optionally via protein-protein intramolecular association forces), protein self-association reactions, and the like. Chromatographic techniques, such as HPLC, can detect the amount of contaminant proteins in a given sample. In exemplary aspects, the protein sample comprises less than about 20% contaminant protein products, less than about 15% contaminant protein products, less than about 10% contaminant protein products, or less than about 5% contaminant protein products, as measured by HPLC-MS. In some aspects, the protein sample comprises less than about 4% contaminant protein products, less than about 3% contaminant protein products, or less than about 2% contaminant protein products, as measured by HPLC-MS.

Additional Preparation and Analysis Steps

The presently disclosed methods may comprise the above described adding step and the above described isolating step alone or in combination with other steps. The methods may comprise repeating any one of the above-described step(s) and/or may comprise additional steps, aside from those described above. For example, the presently disclosed methods may further comprise steps to further process the sample prior to isolating the fraction comprising protein to yield the protein sample. In various instances, the method comprises one or more centrifuging steps to isolate plasma and/or obtain clarified plasma, as described above. In various aspects, the method comprises one or more protein separation steps, e.g., chromatographic, electrophoretic or immunoprecipitation steps. In exemplary aspects, the method comprises depleting the sample of unwanted high concentration proteins, e.g., albumin and/or immunoglobulins. In some aspects, the method comprises one or more of: (a) adding a digestion enzyme, a reducing agent, an alkylating agent, to the sample; (b) identifying proteins present in the sample; (c) quantitating total and individual protein concentration of the sample or an aliquot thereof; and/or (d) labeling proteins or a subset thereof with a tag. Optionally, the digestion enzyme is trypsin. In some aspects, the reducing agent comprises urea or dithiothreitol (DTT) or both. In certain instances, the alkylating agent comprises iodoacetamide (IAA), or a combination thereof.

In various aspects, the method further comprises transporting the mixture in a sealed container to a laboratory for proteomic analysis. Optionally, the sealed container is a sealed BCT comprising the protective agent. In some aspects, the transport to the laboratory requires storing the mixture in the sealed container for at least 24 hours, at least 36 hours, at least 48 hours, at least 60 hours, at least 72 hours, about 84 hours, at least 96 hours or more. In some aspects, the transporting step entails storing the mixture in the sealed container for a storage period for at least about 5 days, at least about 6 days, at least 7 days, or more. In various aspects, the transporting step entails storing the mixture in the sealed container for a storage period at refrigerated temperatures, e.g., about 2° C. to about 8° C., or at temperatures above these temperatures, e.g., about 10° C. to about 15° C., or at an ambient temperature e.g., about 15° C. to about 25° C., about 20° C. to about 25° C. In various aspects, after the transporting step, the mixture is suitable for proteomic analysis as evidenced by the slope of the best fit line of a line graph of the number of proteins in the protein sample yielded from step (b) plotted as a function of storage time being closer to 0 compared to the slope of the best fit line of a line graph of the number of proteins in a control blood sample not contacted with a protective agent and/or the number of plasma proteins and/or peptides present in the protein sample following storage for at least 48 hours being within about 10% of the number of plasma proteins and/or peptides present in the protein sample within about 0 hours to about 4 hours of collecting the blood sample from a subject.

In exemplary aspects, the method prepares a protein sample for proteomic analysis and further comprises carrying out the proteomic analysis. In this regard, the methods may comprise a step of analyzing the proteins using one or more mass spectrometry-based proteomic methods. Suitable methods of proteomic analysis are known in the art, including but not limited to turbidometry, electrophoresis (e.g., capillary electrophoresis, one-dimensional or two-dimensional gel electrophoresis, polyacrylamide gel electrophoresis (PAGE), differential gel electrophoresis (DIGE)), immunoaffinity-based techniques (e.g., Enzyme linked immunosorbent assay (ELISA), sandwich ELISA, competitive ELISA, immunoprecipitation, immunoelectrophoresis, radioimmunoassay), mass spectrometry (e.g., electrospray ionization (ESI)-MS/MS, matrix assisted laser dissociation spectrometry (MALDI)-TOF MS, laser microdissection (LMD)/MS, liquid chromatograph coupled mass spectrometry (LC-MS/MS)), high performance liquid chromatography (HPLC) among other quantitative proteomic techniques (e.g., iTRAQ, ICAT, SILAC), multidimensional protein identification technology (MudPIT), reverse phase protein array, SOMAmers Technology, SELDI, SCX, and the like. See, e.g., Ahmed et al., 2014, supra. In certain aspects, the mass spectrometry-based proteomic methods is a targeted mass spectrometry, In some aspects, the mass spectrometry experiment utilizes parallel reaction monitoring (PRM), selected reaction monitoring (SRM), selected ion monitoring (SIM), or multiple reaction monitoring (MRM). In alternative aspects, the mass spectrometry is a not targeted mass spectrometry. Optionally, the mass spectrometry experiment utilizes data-dependent acquisition (DDA), data independent acquisition (DIA), or labeled quantitation (e.g., tandem mass tag (TMT)) mass spectrometry.

In exemplary aspects, the method prepares a protein sample for proteomic analysis and further comprises carrying out the proteomic analysis and a genomic analysis. Suitable techniques of analyzing the genomic content of a sample are known in the art. See, e.g., Chromosomal Microarray (CMA), linkage analysis, whole exome sequencing (WES), next generation DNA sequences (NGS), and the like.

Storage Stability

Without being bound to a particular theory, the protective agent allows for stabilization of the blood sample, reducing or preventing cell lysis and subsequent release of cellular proteins into the sample. Advantageously, the protein sample isolated from the blood sample is characterized by minimized levels of contaminant protein products, as further described herein. Due to the enhanced stability of the blood sample imparted by the protective agent, the blood sample is capable of longer periods of storage at both refrigerated temperatures and at higher temperatures, e.g., temperatures above 4° C. Surprisingly, the blood sample in contact with the protective agent may be stored for greater than 48 hours and up to 7 days or even longer. The stability allows for the blood sample in contact with the protective agent to be stored for at least 48 hours at 20 QC, for example, and the stability of the blood sample may be evidenced by the low amounts of contaminant protein products after the storage period.

Accordingly, in some aspects, prior to the step of isolating a fraction or cellular fraction, the mixture had been stored for at least 48 hours, for at least 48 hours but less than 7 days or for at least 48 hours but less than 14 days. Optionally, prior to the step of isolating a fraction or cellular fraction, the mixture had been stored for at least 48 hours at a temperature greater than 4° C., optionally, at a temperature of about 20° C. to about 25° C.

Also, accordingly, in various instances, the method comprises storing the mixture prior to the step of isolating a fraction or cellular fraction. In some aspects, the method comprises storing the mixture in the BCT for at least 48 hours, for at least 48 hours but less than 7 days, or for at least 48 hours but less than 14 days prior to the step of isolating a fraction or cellular fraction.

The storage stability of the mixture imparted by the protective agent advantageously allows for proteomic analysis to be carried out on the protein sample at a much later time after the blood sample has been collected, e.g., drawn from the subject. Such storage stability avoids the problems associated with freezing and thawing the protein sample prior to the proteomic analysis.

Reduced Cell Lysis and Contaminating Cellular Proteins Upon Storage

The protective agent allows for stabilization of the blood sample, reducing or preventing cell lysis and subsequent release of cellular proteins into the sample. The protein sample yielded by the presently disclosed methods is advantageously characterized by reduced or decreased cell lysis. While a minimal or base level of cell lysis occurs due to the shear of collecting the blood from the subject, for example, the amount of cell lysis increases over time, e.g., upon storage at refrigerated temperatures or higher temperatures. As a result of the protective agent imparting stability, the protein sample yielded in the method is suitable for proteomic analysis due to the reduced level in cell lysis. In some aspects, the reduced level in cell lysis is a reduction of at least about 10% (e.g., at least about 20%, at least about 30%, at least about 40%, at least about 50%, at least about 60%, at least about 70%, at least about 80% at least about 90%, or more) relative to the level of cell lysis of a control protein sample obtained from an isolated fraction of a blood sample collected in a blood collection tube without a protective agent (e.g., comprising only EDTA). In some aspects, the reduced level in cell lysis is a reduction of at least about 10% (e.g., at least about 20%, at least about 30%, at least about 40%, at least about 50%, at least about 60%, at least about 70%, at least about 80% at least about 90%, or more) relative to the level of cell lysis of a control protein sample obtained from an isolated fraction of a blood sample collected in a blood collection tube without a protective agent (e.g., comprising only EDTA), following storage of the mixture for at least 48 hours prior to the step of isolating the fraction or cellular fraction (optionally, for at least 48 hours but less than 7 days prior to the isolating step, or optionally for at least 48 hours but less than 14 days prior to the isolating step, wherein the storage is at a temperature greater than 4° C., optionally, at a temperature of about 20° C. to about 25° C. Methods of measuring cell lysis are known in the art and include for example, measurement of cell lysis by light scattering (Valenzeno and Trank, Photochemistry and Photobiology 42(3): 335-339 (1985)) and measurement using cell dyes, such as trypan blue.

Reduced cell lysis also may be evidenced by the decrease in contaminating cellular proteins that are released from cells upon cell lysis. In some aspects, the contaminating cellular proteins are cellular proteins from white blood cells, red blood cells, and/or platelets (when the analytes of the proteomic analysis is not proteins of white blood cells, red blood cells, and/or platelets). In some aspects, the protective agent reduces cell lysis of white blood cells, red blood cells, and/or platelets so that there is a reduced level of contaminating cellular proteins from these cells. While a minimal or base level of contaminating cellular proteins may be present, due to the shear of collecting the blood from the subject, for example, the amount of contaminating cellular proteins increases over time, e.g., upon storage at refrigerated temperatures or higher temperatures. As a result of the protective agent imparting stability, the protein sample yielded in the method is suitable for proteomic analysis due to the reduced level in contaminating cellular proteins. In some aspects, the reduced level in contaminating cellular proteins is a reduction of at least about 10% (e.g., at least about 20%, at least about 30%, at least about 40%, at least about 50%, at least about 60%, at least about 70%, at least about 80% at least about 90%, or more) relative to the level of contaminating cellular proteins of a control protein sample obtained from an isolated fraction of a blood sample collected in a blood collection tube without a protective agent (e.g., comprising only EDTA). In some aspects, the reduced level in contaminating cellular proteins is a reduction of at least about 10% (e.g., at least about 20%, at least about 30%, at least about 40%, at least about 50%, at least about 60%, at least about 70%, at least about 80% at least about 90%, or more) relative to the level of contaminating cellular proteins of a control protein sample obtained from an isolated fraction of a blood sample collected in a blood collection tube without a protective agent (e.g., comprising only EDTA), following storage of the mixture for at least 48 hours prior to the step of isolating the fraction or cellular fraction (optionally, for at least 48 hours but less than 7 days prior to the isolating step, or optionally for at least 48 hours but less than 14 days prior to the isolating step, wherein the storage is at a temperature greater than 4° C., optionally, at a temperature of about 20° C. to about 25° C. Methods of measuring contaminating cellular proteins are known in the art and include for example, measurement of a representative contaminating cellular protein. In some aspects, the representative contaminating cellular protein is a protein of the red blood cell proteome, described in Pasini et al., Blood 108(3): 791-801 (2006) or Bryk and Wisniewski, J Proteome Res 16: 2752-2761 (2017). In some aspects, the representative contaminating cellular protein is hemoglobin, or a subunit thereof (HbA, HbB, HbD, HbG, HbZ), or carbonic anhydrase (CA1), or a peroxiredoxin (e.g., PRDZ1, PRDX12, PRDX16), biliverdin reductase B (BLVRB), catalase (CAT), superoxide dismutase (SOD1), bisphosphoglycerate mutase (BPGM). In some aspects, the representative contaminating cellular protein is a protein of the white blood cell proteome, e.g., leukocyte-specific protein 1 (LSP1), a subunit of the T-cell receptor, a subunit of the B-cell receptor. In some aspects, the representative contaminating cellular protein is a protein of the platelet proteome, such as those described in Senzel et al., Curr Opin Hematol 16(5): 329-333 (2009) and Doyle et al., Blood J 55(1): 82-84. In some aspects, the representative contaminating cellular protein is beta-thromboglobulin, and platelet factor 4.

Levels of cell lysis may also be measured by measuring cell stabilization, as represented by cell-free DNA using droplet digital PCT (ddPCR). See, e.g., Norton et al., Clin Biochem 46: 1561-1565 (2013).

In various aspects, the low levels of cell lysis may be evident from the slope of the best fit line of a line graph of the number of proteins in the protein sample yielded from step (b) plotted as a function of storage time. In exemplary aspects, the slope of the best fit line of a line graph of the number of proteins in the protein sample yielded from step (b) plotted as a function of storage time is closer to 0 compared to the slope of the best fit line of a line graph of the number of proteins in a control blood sample not contacted with a protective agent.

In various aspects, the low levels of cell lysis may be evident from the slope of the best fit line of a line graph of the number of peptides in the protein sample yielded from step (b) plotted as a function of storage time. In exemplary aspects, the slope of the best fit line of a line graph of the number of peptides in the protein sample yielded from step (b) plotted as a function of storage time is closer to 0 compared to the slope of the best fit line of a line graph of the number of peptides in a control blood sample not contacted with a protective agent.

Plasma Fractions and Increased Levels of Low Abundance Plasma Proteins or Unique Peptides per Protein or Unique Proteins Identified Upon Storage

In some aspects, the fraction isolated from the mixture is a plasma fraction and the protein sample yielded in the method is suitable for proteomic analysis due to an increased level of low-abundance plasma proteins. In some aspects, the increased level of low-abundance plasma proteins is an increase of at least about 10% (e.g., at least about 20%, at least about 30%, at least about 40%, at least about 50%, at least about 60%, at least about 70%, at least about 80% at least about 90%, or more) relative to the level of low-abundance plasma proteins present in a control protein sample obtained from an isolated fraction of a blood sample collected in a blood collection tube without a protective agent (e.g., comprising only EDTA). In some aspects, the increased level of low-abundance plasma proteins is an increase of at least about 10% (e.g., at least about 20%, at least about 30%, at least about 40%, at least about 50%, at least about 60%, at least about 70%, at least about 80% at least about 90%, or more) relative to the level of low-abundance plasma proteins present in a control protein sample obtained from an isolated fraction of a blood sample collected in a blood collection tube without a protective agent (e.g., comprising only EDTA), following storage of the mixture for at least 48 hours prior to the step of isolating the fraction or cellular fraction (optionally, for at least 48 hours but less than 7 days prior to the isolating step, or optionally for at least 48 hours but less than 14 days prior to the isolating step, wherein the storage is at a temperature greater than 4° C., optionally, at a temperature of about 20° C. to about 25° C. Methods of measuring levels of low-abundance plasma proteins are known in the art. See e.g., Liu et al., PLOS One DOI:10.1371/journal.pone.0166306 (2016), which also describes over 125 low-abundance plasma proteins.

In some instances, the fraction isolated from the mixture is a plasma fraction and the protein sample yielded in the method is suitable for proteomic analysis due to an increased level of unique peptides identified per protein. In exemplary instances, the unique peptides identified per protein are determined by discovery-label-free data dependent acquisition (DDA) LC-MS/MS. In some aspects, the increased level of unique peptides identified per protein is an increase of at least about 10% (e.g., at least about 20%, at least about 30%, at least about 40%, at least about 50%, at least about 60%, at least about 70%, at least about 80% at least about 90%, or more) relative to the level of unique peptides identified per protein present in a control protein sample obtained from an isolated fraction of a blood sample collected in a blood collection tube without a protective agent (e.g., comprising only EDTA). In some aspects, the increased level of unique peptides identified per protein is an increase of at least about 10% (e.g., at least about 20%, at least about 30%, at least about 40%, at least about 50%, at least about 60%, at least about 70%, at least about 80% at least about 90%, or more) relative to the level of unique peptides identified per protein present in a control protein sample obtained from an isolated fraction of a blood sample collected in a blood collection tube without a protective agent (e.g., comprising only EDTA), following storage of the mixture for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. Methods of measuring the level of unique peptides identified per protein may be carried out by DDA LC-MS/MS as essentially described in Almazi et al., Proteomics Clin Applications 12: 1700121 (2018); doi: 10.1002/prca.201700121.

In some instances, the fraction isolated from the mixture is a plasma fraction and the protein sample yielded in the method is suitable for proteomic analysis due to an increased level of unique proteins identified, as determined by LC-MS/MS, optionally, wherein the unique proteins are secretory proteins. In some aspects, the increased level of unique proteins identified is an increase of at least about 10% (e.g., at least about 20%, at least about 30%, at least about 40%, at least about 50%, at least about 60%, at least about 70%, at least about 80% at least about 90%, or more) relative to the level of unique proteins identified present in a control protein sample obtained from an isolated fraction of a blood sample collected in a blood collection tube without a protective agent (e.g., comprising only EDTA). In some aspects, the increased level of unique proteins identified is an increase of at least about 10% (e.g., at least about 20%, at least about 30%, at least about 40%, at least about 50%, at least about 60%, at least about 70%, at least about 80% at least about 90%, or more) relative to the level of unique proteins identified present in a control protein sample obtained from an isolated fraction of a blood sample collected in a blood collection tube without a protective agent (e.g., comprising only EDTA), following storage of the mixture for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. Methods of measuring the level of unique proteins identified may be carried out by LC-MS/MS as essentially described in Tsung-Heng Tsai et al., Proteomics 15(13): 2369-2381 (2015) and Geyer et al., Cell Systems 2: 185-195 (2016).

Likeness to Freshly Isolated Blood Sample

In some aspects, the protein sample yielded in the method is suitable for proteomic analysis due its likeness to a freshly isolated blood sample, in terms of intact protein content, even after the protein sample has been stored. As used herein, the term “freshly isolated” in “freshly isolated blood sample” refers to a blood sample wherein not more than 26 hours has passed since the time the blood sample was isolated, collected or drawn from a subject. In various instances, the protein sample yielded in the method is suitable for proteomic analysis as the protein sample comprises greater than about 50%, greater than about 60% (e.g., greater than about 70%, greater than about 80%, greater than about 90% or more) of the intact proteins present in a freshly isolated blood sample. In various instances, the protein sample yielded in the method is suitable for proteomic analysis as the protein sample comprises greater than about 50%, greater than about 60% (e.g., greater than about 70%, greater than about 80%, greater than about 90% or more) of the intact proteins present in a freshly isolated blood sample, even following storage of the protein sample for at least 48 hours prior to the step of isolating the fraction or cellular fraction (optionally, for at least 48 hours but less than 7 days prior to the isolating step, or optionally for at least 48 hours but less than 14 days prior to the isolating step, wherein the storage is at a temperature greater than 4° C., optionally, at a temperature of about 20° C. to about 25° C. Methods of measuring intact protein content are known in the art and include for example SDS-PAGE and mass spectrometry.

Also, in various instances, the likeness to a freshly isolated blood sample may be evident from the number of plasma proteins and/or peptides present in the protein sample following storage for at least 48 hours being very similar to the number of plasma proteins and/or peptides present in the protein sample within about 0 hours to about 4 hours of collecting the blood sample from a subject. In various instances, the number of plasma proteins and/or peptides present in the protein sample following storage for at least 48 hours is within about 20% or about 25% of the number of plasma proteins and/or peptides present in the protein sample within about 0 hours to about 4 hours of collecting the blood sample from a subject. In various instances, the number of plasma proteins and/or peptides present in the protein sample following storage for at least 48 hours is within about 10% of the number of plasma proteins and/or peptides present in the protein sample within about 0 hours to about 4 hours of collecting the blood sample from a subject. In various instances, the number of plasma proteins and/or peptides present in the protein sample following storage for at least 48 hours is within about 7.5% or about 5% of the number of plasma proteins and/or peptides present in the protein sample within about 0 hours to about 4 hours of collecting the blood sample from a subject.

Reduced Contaminating Protein Products

In some aspects, the protein sample yielded in the method is suitable for proteomic analysis due its reduced level of contaminating protein products, relative to the level of contaminating protein products in a control protein sample obtained from an isolated fraction of a blood sample collected in a blood collection tube without a protective agent (e.g., comprising only EDTA), following storage of the blood sample from which the protein sample or control sample derived for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. In exemplary instances, the protein sample yielded in the method comprises less than about 40%, (e.g., less than about 35%, less than about 30%, less than about 25%, less than about 20%, less than about 15%, less than about 10%, or less than about 5%) of the contaminating protein products, relative to the level of contaminating protein products in a control protein sample obtained from an isolated fraction of a blood sample collected in a blood collection tube without a protective agent (e.g., comprising only EDTA), following storage of the blood sample from which the protein sample or control sample derived for at least 2 hours prior to the step of isolating the fraction or cellular fraction (optionally, for at least 2 hours but less than 4 hours prior to the isolating step, or optionally for at least 2 hours but less than 8 hours prior to the isolating step, wherein the storage is at a temperature greater than 4° C., optionally, at a temperature of about 20° C. to about 25° C. As used herein “contaminating protein products” refer to oxidized, reduced, amidated, deamidated, lysed, degraded, aggregated, and/or precipitated protein products. Methods of measuring contaminating protein products are known in the art and include for example HPLC and MS.

The following examples are given merely to illustrate the present invention, and the advantages thereof, and not in any way to limit its scope.

### EXAMPLES

### Example 1

This example describes the proteomic analysis of plasma and depleted-plasma from whole blood samples collected in two different types of blood collection tubes (BCTs).

The objective of this assay was to process human whole blood samples using different blood collection tubes (BCTs) to obtain plasma and then analyze by LC-MS/MS. Tube 1 was a control EDTA blood collection tube lacking a protective agent. Tube 1 contained a liquid additive EDTA (K3) 15% solution; and Tube 2 was a BCT comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA.

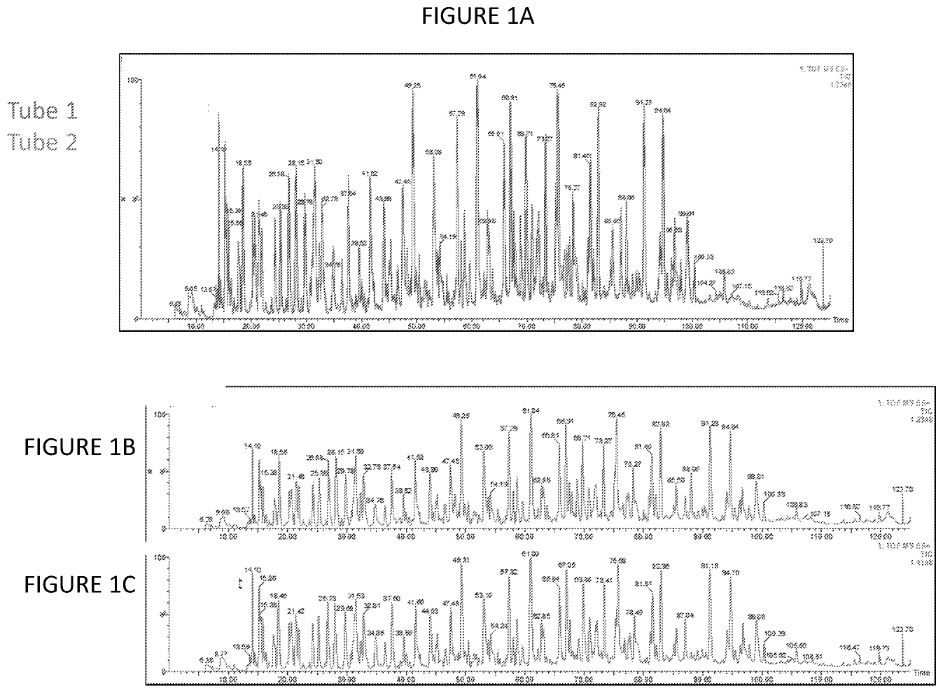

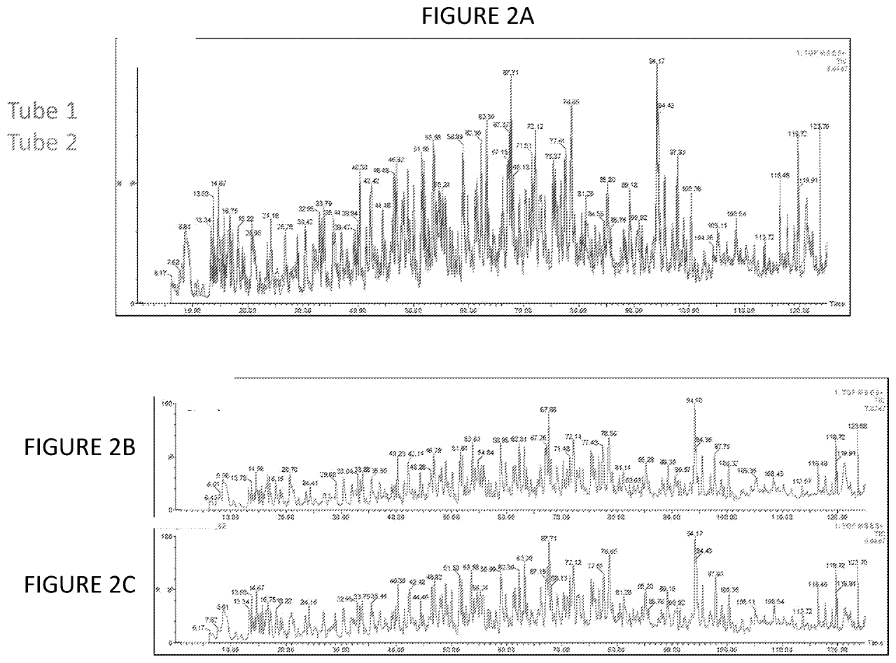

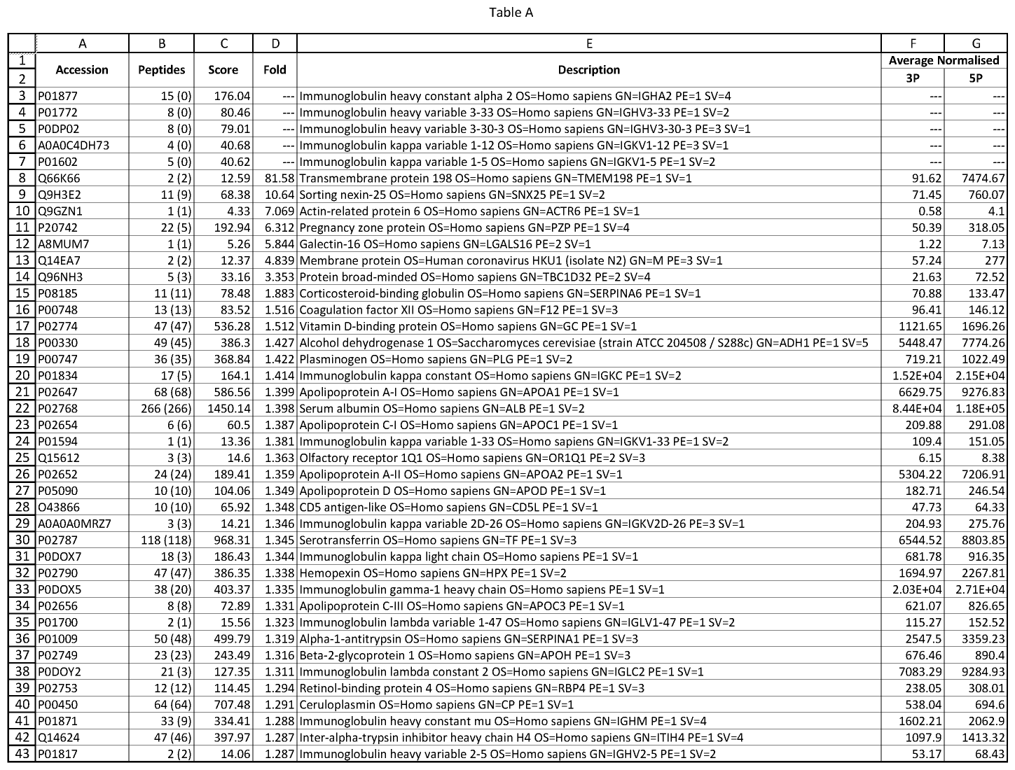

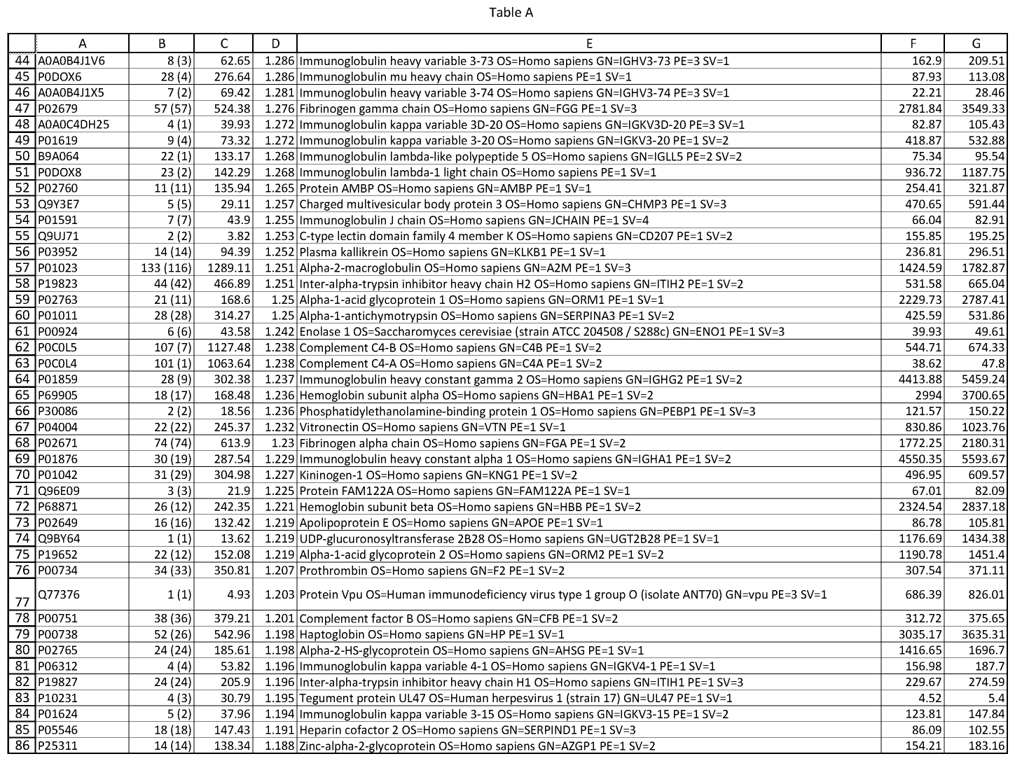

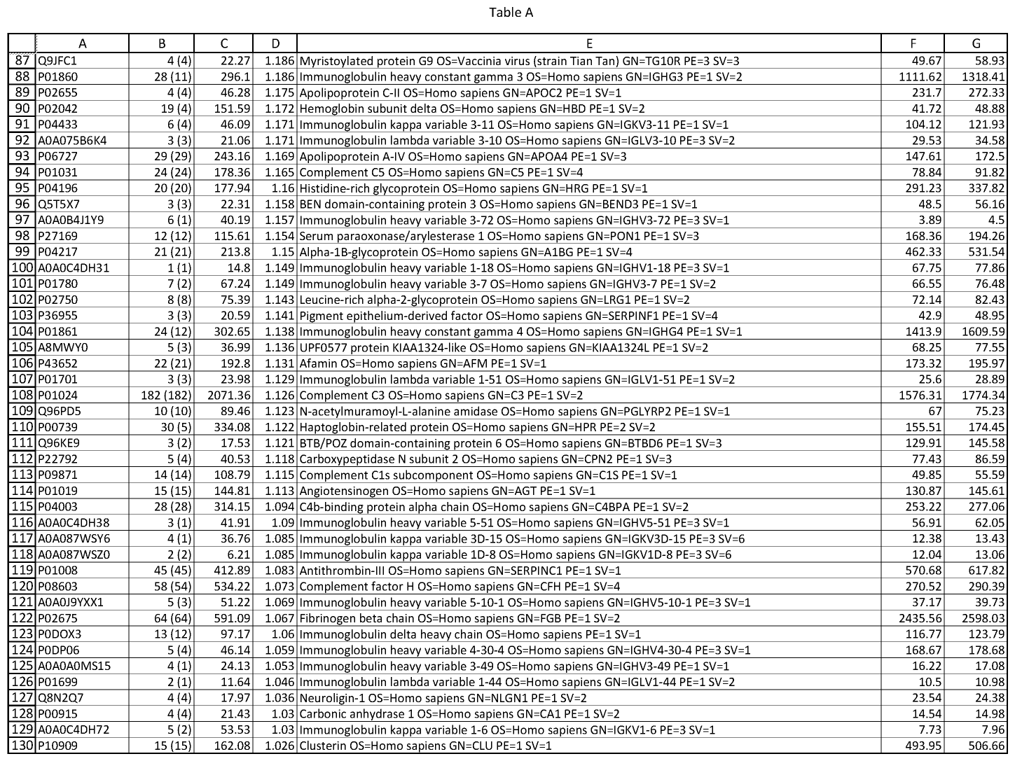

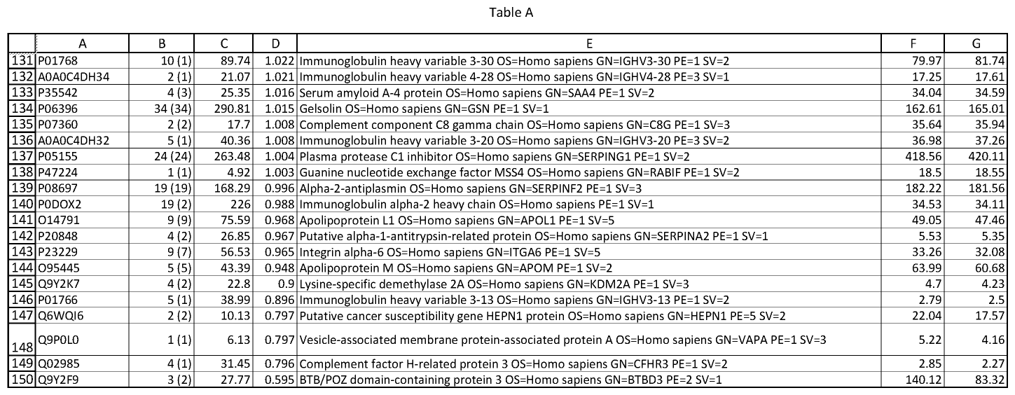

Proteomics analysis on whole plasma and depleted plasma samples derived from human whole blood collected in different BCTs was carried out via LC-MS to obtain total ion chromatogram (TIC) plots. Details of the LC-MS runs are provided in Table 3. The TIC for whole plasma samples are shown in FIGS. 1A-1C, and the TIC for depleted plasma samples are shown in FIGS. 2A-2C. FIG. 1A provides an overlay TIC plot of the Tube 1 whole plasma sample (red trace) and the Tube 2 whole plasma sample (green trace). FIG. 1B and FIG. 1C are stacked view of the Tube 1 whole plasma sample (FIG. 1B) and the Tube 2 whole plasma sample (FIG. 1C). FIG. 2A provides an overlay TIC plot of the Tube 1 depleted plasma sample (red trace) and the Tube 2 depleted sample (green trace). FIGS. 2B and 2C are stacked view of the Tube 1 depleted sample (FIG. 2B) and the Tube 2 depleted sample (FIG. 2C).

As shown in FIGS. 2A-2C, the chromatograms of depleted plasma samples derived from human whole blood collected in Tube 1 and Tube 2 are about the same. A total of 153 proteins were identified in the plasma. Seven proteins had a fold change >2 between tubes, protein abundance was greater in Tube 2 for these proteins. Proteins with higher abundances in the Tube 2 were all known plasma proteins.

Progenesis Reports

Progenesis Identifies Proteins in the Sample and Compares the Relative Abundance of Proteins Among the Samples

The results are provided in Tables A and B, wherein Table A provides information from whole plasma and Table B provides information from depleted plasma samples. In each table, the following descriptions apply:

Peptides: Total number of peptides detected from the protein. The number in the parentheses is the number of unique peptides detected from the protein. Some tryptic peptides detected in the experiment are common to multiple proteins. Proteins with a high degree of homology may have shared tryptic peptides.

Score: The score Progenesis uses to quantify the goodness and reliability of the protein identification. The higher the score, the more reliable the identification.

Fold: This is a measure of the maximum difference in relative abundance of the protein among the samples. This should be regarded as an estimate. Note that yeast ADH was spiked at equal amounts into each sample, and a maximum fold difference of 1.55 was obtained.

Average Normalised Abundances: The protein level Progenesis calculated for each sample. This is calculated by Progenesis based on the signal intensity from the 3 most abundant peptides found for each protein and with respect to the level of spiked yeast ADH.

Discussion: In the whole plasma samples, albumin and immunoglobulins were the most abundant proteins in the samples, as expected. In depleted plasma samples, albumin and immunoglobulins were still detected by at much lower levels, thus signaling the success of the albumin and immunoglobulin depletion. Depletion also appeared consistent among all samples analyzed.

In the whole plasma samples, the majority of the proteins were at similar relative levels. Of the 148 proteins in the list, 7 had “Fold”>2. The protein profile looked very similar among the collection tubes.

In depleted plasma samples, the majority of the proteins were at similar relative levels. Of the 153 proteins in the list, only 7 had “Fold”>2. Given the depletion processing steps carried out, the protein profile looked very similar among the collection tubes.

This example demonstrated that, in this analysis, the protein profiles between the collection tube samples were very similar and the stabilizing reagent is suitable for proteomic analyses.

### Example 2

This example describes the materials and methods used in the analysis of Example 1.

Samples were transported to an analysis laboratory on ice and stored at 4° C. until further analysis. Plasma was extracted from whole blood samples by centrifugation. A portion of the isolated plasma was treated with Albumin and IgG Depletion SpinTrap Columns (GE Health Care), depleting the plasma of albumin and IgG. Whole plasma and depleted plasma were processed by reduced tryptic digestion. A full description of these steps follows.

Materials

Human whole blood was collected in one of the types of blood collection tubes (BCT) tested: Tube 1 was a control EDTA blood collection tube lacking a protective agent. Tube 1 contained a liquid additive EDTA (K3) 15% solution; and Tube 2 was a BCT comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA. Proteolytic enzyme inhibitors were not used in the collection of blood or any aspect of this study.

Plasma Isolation from Human Whole Blood

Plasma was isolated from human whole blood by carrying out the following steps: (1) Per sample. 1 mL of whole blood was added to a 1.5 mL Protein LoBind Tubes. Samples were centrifuged at room temperature in an Eppendorf Centrifuge 5415D at 2000 g for 15 minutes. The plasma supernatant was removed an added to a new 1.5 mL Protein LoBind Tube. (2) Plasma was centrifuged again at room temperature in an Eppendorf Centrifuge 5415D at 16000 g for 10 minutes. The plasma supernatant was removed an added to a new 1.5 mL Protein LoBind Tube. (3) Clarified plasma was stored on ice. After processing, depleted plasma was stored at −80° C. until further analysis.

Pre-Treatment of Isolated Plasma Using Albumin & IgG Depletion Spin Trap Columns

Albumin & IgG were depleted from the isolated plasma by carrying out the following steps: (1) Per sample, 2504 of a 1:1 dilution of clarified plasma in 20 mM Sodium phosphate, 0.15M Sodium chloride, pH 7.4 was prepared. 125 μL of clarified plasma and 125 μL 20 mM Sodium phosphate, 0.15M Sodium chloride, pH 7.4 were combined in a 0.5 mL Protein LoBind Tube and gently vortexed using a Vortex Genie 1 to mix. (2) Six Albumin & IgG Depletion SpinTrap columns were inverted repeatedly to suspend the medium. (3) The bottom cap from the SpinTrap columns was removed and the top cap turned one-quarter of a turn. The SpinTrap columns were placed in 1.5 mL Protein LoBind Tubes and centrifuged at room temperature for 30 seconds at 800 g in an Eppendorf Centrifuge 5415D. Flow through was discarded. Top caps were discarded and SpinTrap columns placed back in 1.5 mL Protein LoBind Tubes. (4) 4004 of 20 mM Sodium phosphate, 0.15M Sodium chloride, pH 7.4 (binding buffer) was added to the medium. SpinTrap columns were centrifuged at room temperature for 30 seconds at 800 g in an Eppendorf Centrifuge 5415D. Flow through was discarded. Step was repeated. (5) SpinTrap columns were placed into new 1.5 mL Protein LoBind Tubes. Per sample, 1004 of diluted clarified plasma was added to two equilibrated SpinTrap medium. Samples were incubated at room temperature for 5 minutes. (6) SpinTrap columns were centrifuged at room temperature for 30 seconds at 800 g in an Eppendorf Centrifuge 5415D, collecting the eluate. 1004 of binding buffer was added to the SpinTrap medium. SpinTrap columns were centrifuged at room temperature for 30 seconds at 800 g in an Eppendorf Centrifuge 5415D, collecting the eluate. Step was repeated. Note: All eluates were collected in the same 1.5 mL Protein LoBind Tube. (7) Sample eluates were combined into one 1.5 mL Protein LoBind Tube and OD280 nm analysis performed on the IgG and albumin depleted serum. (8) After processing, depleted plasma was stored at −80° C. until further analysis.

OD280 nm Analysis of IgG and Albumin Depleted Serum

Protein concentration of IgG- and Albumin-depleted plasma was determined by the carrying out the following steps: (1) The protein concentration of depleted plasma and clarified plasma was determined by OD280 nm using a Beckman Coulter Du520 General Purpose UV/VIS spectrophotometer and a Spectrophotometer Cell UV, Micro, Black Walled quartz cuvette (VWR, Cat. #58016-505), 10 mm light path. (2) Prior to A280 nm analysis, the instrument was zeroed by taking a blank reading of 20 mM Sodium phosphate, 0.15M Sodium chloride, pH 7.4. 4004 of 20 mM Sodium phosphate, 0.15M Sodium chloride, pH 7.4 was added to the quartz cuvette, which was sufficient volume to cover the aperture through which the light beam passed. (3) Absorbance measurement of the depleted plasma at 280 nm was carried out by filling the cuvette with 400 μL of a 10× dilution, where 504 of sample was diluted into 450 μL 20 mM Sodium phosphate, 0.15M Sodium chloride, pH 7.4. (4) Absorbance measurement of the clarified plasma at 280 nm was carried out by filling the cuvette with 400 μL of a 100× dilution, where 10 μL of sample was diluted into 990 μL 20 mM Sodium phosphate, 0.15M Sodium chloride, pH 7.4. (5) Between sample reads, the cuvette was rinsed 3 times with methanol and air-dried. (6) The extinction coefficient of 1.0 (mg/mL)⁻¹ (cm)⁻¹ was chosen for the purpose of this assay. (7) Protein concentration, c, was determined using the Beer-Lambert Law: A (280 nm)=εcl, where ε=extinction coefficient of 1.0 (mg/mL)⁻¹ (cm)⁻¹ and l=optical path length, where in cm, which was equivalent to 1 cm.

Reduction, Alkylation, DTT Quench and Trypsin Digestion, pH 7.8

Prior to trypsin digestion, clarified plasma was diluted to a concentration of 10 mg/mL in HPLC grade water. 300 pmoles of each sample was reduced with 5 mM Dithiotheitol, 50 mM Ammonium bicarbonate, pH 7.8, 60° C. for 1 hour. After reduction, sample was alkylated with 15 mM Iodoacetamide, 50 mM Ammonium bicarbonate, pH 7.8 at room temperature, in the dark for 30 minutes. After alkylation, the sample was quenched with 5 mM DTT, 50 mM Ammonium bicarbonate, pH 7.8, and incubated at room temperature for 30 minutes. The sample was digested with Trypsin using a 1:50 enzyme to substrate ratio, at 37° C., for 18 hours. The digest was quenched to a final concentration of 0.5% formic acid and stored at −80° C. until further analysis.

Liquid Chromatography-Mass Spectrometry (LC-MS) Analysis

Waters MassPrep Enolase (ENOL) and ADH digests were spiked into the samples as detailed in Table 1:

TABLE 1

| | |
|---|---|
| ADH | use straight; spike in 2 μL to the final |
| preparation notes | sample in the LC vial |
| ENOL | dilute 25x prior to spiking; spike in 1 uL; |
| preparation notes | 48 uL Solvent A adjusted to 5% ACN with |
| | ACN + 2 uL digest |
| | 45.5 μL Solvent A |
| | 2.5 μL 100% ACN |
| | 2.0 μL ENOL digest |
| | Spike in 1 uL into the LC vial |
| | 50-fold difference in amounts |
| Amount Injected (per | ADH = ~6.8 pmol |
| sample) | ENOL = ~135 fmol |

Digests were thawed in a water bath set at room temperature and then centrifuged in a benchtop centrifuge for ˜10 seconds to settle. ENOL dilution was made into Axygen lo-binding microtubes. Each sample was added to Dionex Polypro 300 uL LC vial, and then add requisite amounts of MassPrep digests. Table 2 provides details on the column, solvents and gradient program used, and Table 3 provides details of the LC-MS runs.

TABLE 2

| | |
|---|---|
| Column: | Dionex C18 Pepmap, 1 mm × 150 mm |
| Solvent A: | 0.1% formic acid in water |
| Solvent B: | 0.1% formic acid in acetonitrile |
| Gradient program | 123 min MS acquisition |
| for samples | Oven at 60 C., WPS at 6 C. |
| | 0-30% B at 0.3%/min |
| | 30-40% B at 1%/min |
| | Then cleaning sawtooth |
| | Clean with 50 uL IPA injection after sample |
| | Re-equilibrate with 100 uL ACN injection after |
| | IPA injection |
| Gradient program | 70 min MS acq |
| for BSA injections | |
| done for system | |
| suitability - | |
| shorter method | |

TABLE 3

| Digest #1 | | Digest #2 | |
|---|---|---|---|
| Conc ADH MP std [pmol/uL] - not diluted | 4 | Conc ADH MP std [pmol/uL] - not diluted | 4 |
| Conc Enolase MP std [pmol/uL] - diluted 25x | 0.16 | Conc Enolase MP std [pmol/uL] - diluted 25x | 0.16 |
| Conc SAMPLE digest [ug/uL] | 0.7 | Conc SAMPLE digest [ug/uL] | 0.5 |
| SAMPLE digest Aliquot Size [uL] | 30 | SAMPLE digest Aliquot Size [uL] | 29 |
| Vol SAMPLE Digest [uL] - Set | 30.0 | Vol SAMPLE Digest [uL] - Set | 29.0 |
| Vol ADH MP-Std digest [uL] | 2.0 | Vol ADH MP-Std digest [uL] | 2.0 |
| Vol Enolase MP-Std Digest [uL] | 1.0 | Vol Enolase MP-Std Digest [uL] | 1.0 |
| Total Vol | 33 | Total Vol | 32 |
| Conc SAMPLE digest in vial [ug/uL] | 0.636 | Conc SAMPLE digest in vial [ug/uL] | 0.453 |
| Conc ADH MP-Std in vial [pmol/uL] | 0.242 | Conc ADH MP-Std in vial [pmol/uL] | 0.250 |
| Conc Enolase MP-Std in vial [pmol/uL] | 0.0048 | Conc Enolase MP-Std in vial [pmol/uL] | 0.0050 |
| Total Vol Injected [uL] | 28 | Total Vol Injected [uL] | 27 |
| Amt SAMPLE Injected [ug] | 17.82 | Amt SAMPLE Injected [ug] | 12.23 |
| Amt ADH MP-Std injected [pmol] | 6.79 | Amt ADH MP-Std injected [pmol] | 6.75 |
| Amt Enolase MP-Std injected [pmol] | 0.136 | Amt Enolase MP-Std injected [pmol] | 0.135 |

Data Analysis

Raw data were searched against a UniProt human proteome database. This database contained only reviewed proteins from UniProt. Yeast ADH and yeast enolase were added manually. Data were searched in Progenesis QI (Waters) and PLGS (Waters); both of these programs have the same underlying search engine for MS-E data.

### Example 3

This example demonstrates a proteomic analysis of plasma and depleted-plasma obtained from whole blood collected in BCTs comprising a protective agent with and without proteolytic enzyme inhibitors.

Whole blood samples are collected in one of two types of BCTs: BCTs with a protective agent or BCTs without a protective agent. Plasma is extracted from whole blood samples by centrifugation. For one series, the isolated plasma is treated with Albumin and IgG Depletion SpinTrap Columns (GE Health Care), depleting the plasma of albumin and IgG. For another series, depletion is not carried out. All plasma samples (depleted or not depleted) are further processed by reduced tryptic digestion followed by LC/MS proteomic analysis. In one half of the test samples, a solution comprising cOmplete™, Mini, EDTA-free protease inhibitor cocktail (PIC) tablets (Roche brand, commercially available from Sigma-Aldrich, St. Louis, Mo.) is added. All samples (depleted and undepleted plasma samples; with or without PIC) are proteomic-analyzed via LC-MS as essentially described above. These procedures are carried out as essentially described in Example 2

It is expected that the TIC overlay of the samples without the PIC and the samples with the PIC are about the same, demonstrating that the use of PIC and like proteolytic enzyme inhibitors in methods of preparing samples for proteomic analysis is unnecessary.

### Example 4

This example demonstrates the stability and suitability for proteomic analysis of samples derived from whole blood collected in BCTs with or without a protective agent without proteolytic enzyme inhibitors following storage at room temperature for extended time periods.

As discussed above, reduced cell lysis may be measured as cell stability which in turn may be indirectly measured by quantifying the amount of cell-free DNA by droplet digital PCR. Whole blood samples are collected in one of two types of BCTs: BCTs with a protective agent or BCTs without a protective agent. In a first set of experiments, a solution comprising cOmplete™, Mini, EDTA-free protease inhibitor cocktail (PIC) tablets (Roche brand, commercially available from Sigma-Aldrich, St. Louis, Mo.) is added. In a second set of experiments, the solution comprising PIC tablets is not used. Samples are stored at room temperature for varying times: 1 hour, 12 hours, 24 hours, 48 hours, 72 hours, 120 hours, 240 hours, 336 hours (2 weeks), 1 month, 2 months, 4 months and 6 months. Cell free-DNA present in each sample is measured by DD-PCR, as essentially described in Norton et al., 2013, supra. Additional measurements and observations of each sample are taken, including, e.g., overall protein concentration as determined by UV-VIS and estimation of intact protein content as determined by SDS-PAGE. Each sample is subjected to processing steps as essentially described in Example 2 and mass spectrometry based proteomic analysis following the procedures described in Example 2 or in Almazi et al., 2018, supra are carried out.

It is expected that the levels of cell free DNA are lower and intact protein content is higher in the samples derived from whole blood samples collected in BCTs with a protective agent, compared to the level of cell free DNA present in the samples from whole blood samples collected in BCTs without a protective agent. Among the samples derived from whole blood samples collected in BCTs with a protective agent, it is expected that the levels of cell free DNA and intact protein content are approximately the same, suggesting that the use of PIC tablets in the preparation of protein samples is not needed.

Furthermore, it is expected that the level of unique proteins identified per protein as determined by the LC/MS methodology of Almazi et al., 2018, supra is about the same among samples derived from whole blood samples collected in BCTs with a protective agent, regardless of whether a solution containing PIC tablets was added, suggesting that the use of PIC tablets in the preparation of protein samples is not needed.

### Example 5

This example demonstrates an exemplary method of preparing a protein sample for proteomic analysis using one or more mass spectrometry-based proteomic methods, wherein the mass spectrometry of the mass spectrometry-based proteomic methods is a targeted mass spectrometry. This example demonstrates that collection of blood using CF BCTs, which comprises a protective agent, successfully provided protein samples that were stable even after storage for up to 216 hours.

Human plasma samples were collected using one of two types of blood collection tubes (BCTs). “E BCTs” were control EDTA BCTs lacking a protective agent and containing a liquid additive EDTA (K3) 15% solution. “CF BCTs” were BCTs comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA.

Blood samples were drawn directly into a BCT from the donor via venipuncture following the CLSI Approved Standard “Procedures for the Collection of Diagnostic Blood Specimens by Venipuncture”, CLSI Document GP41. Samples were collected in biological replicates corresponding to three individuals. The samples were stored for 0 hours up to 216 hours at ambient temperature, e.g., about 15° C. to about 25° C. temperature (about 20° C. to about 25° C.).

Samples were prepared for mass spectrometry by reducing and alkylating proteins and digesting with trypsin. C18 purification of peptides and quantification of final peptide concentration were carried out as described in “Sample Preparation Kit Pro: For High-Throughput Mass Spectrometry Proteomics” Manual, First Edition, Version 1.04 (November 2017), Biognosys AG, Switzerland at https://www.biogonosys.com/media/5c169d9c-50fe-4d7b-84a4-a1dec78f6a63.pdf. Final peptide preparations were spiked with the PQ500™ panel of stable isotope standard (SIS) peptides.

HRM LC-MS/MS was carried out for protein profiling and HRM maps were recorded. Data was extracted using the PQ500™ panel and the data were analyzed by a number of methods, including QC metrics, principle component analysis (PCA), and partial least squares discriminant analysis (PLS-DA). The absolute peptide and protein quantities were calculated. All data were statistically tested.

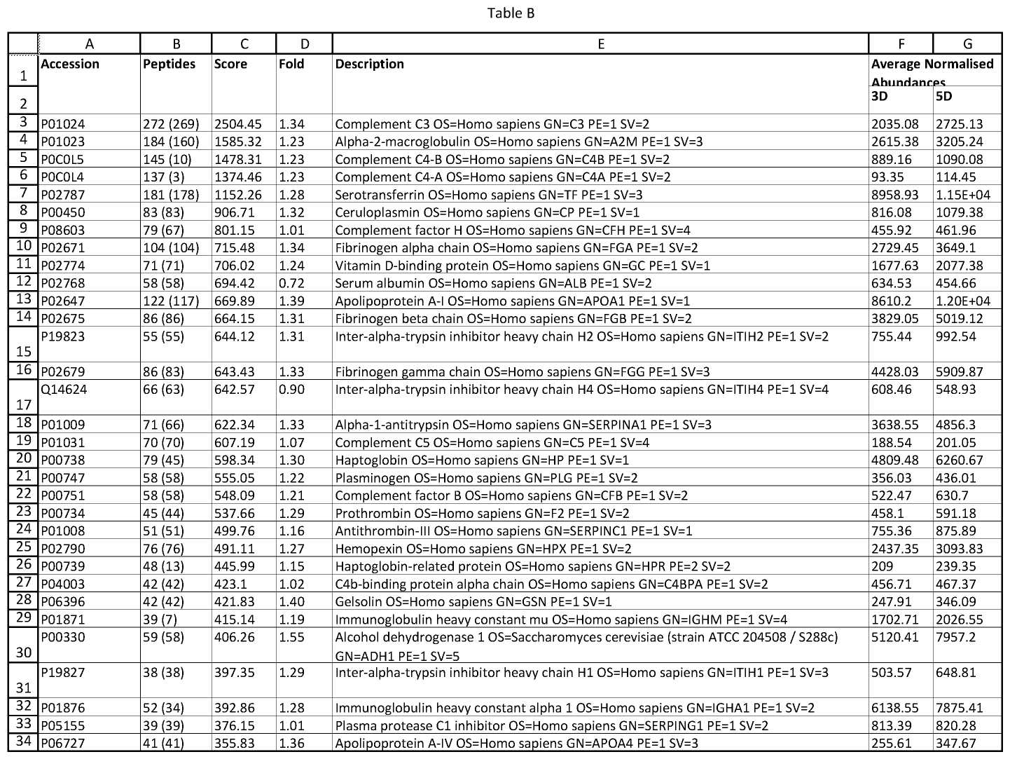

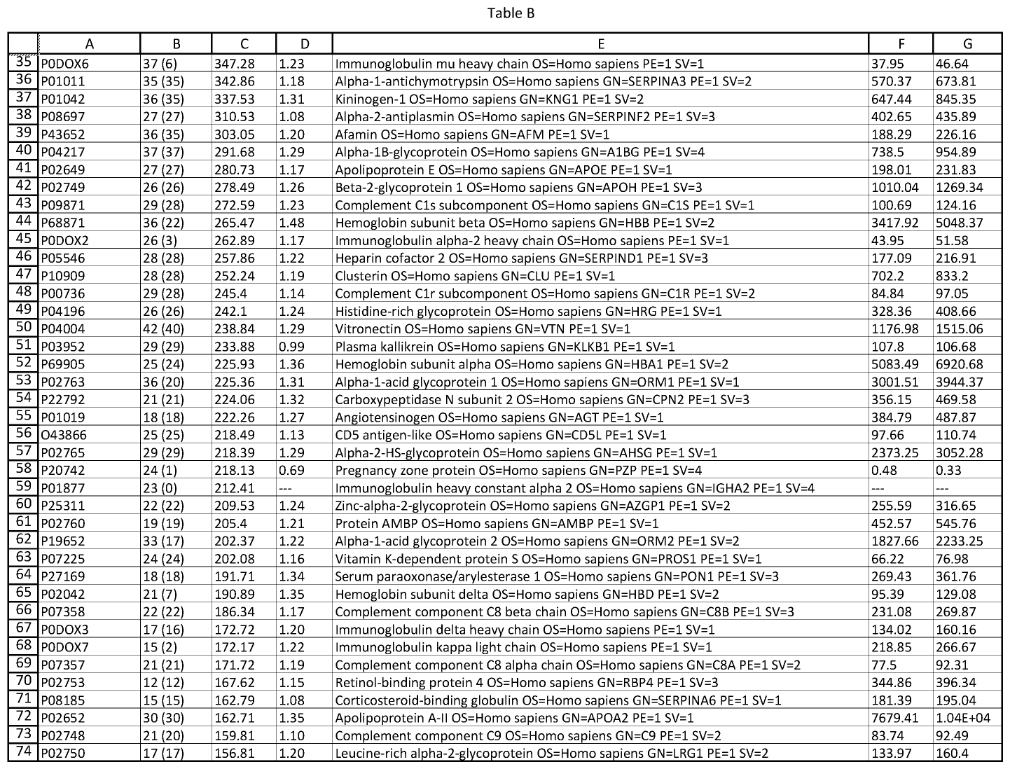

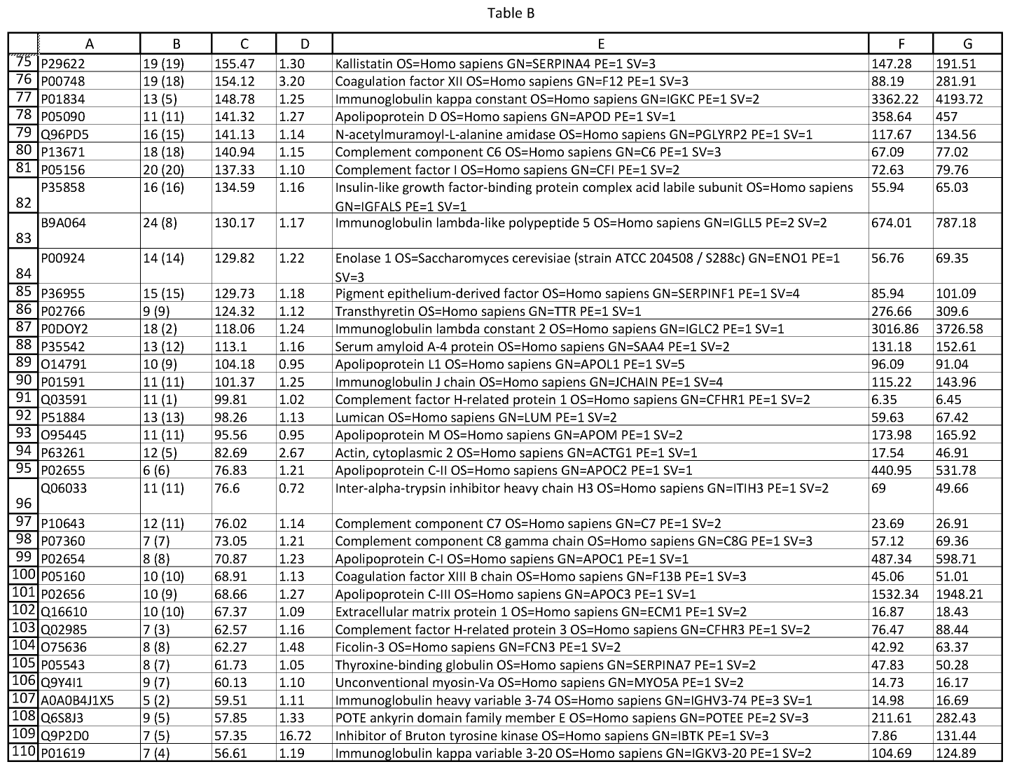

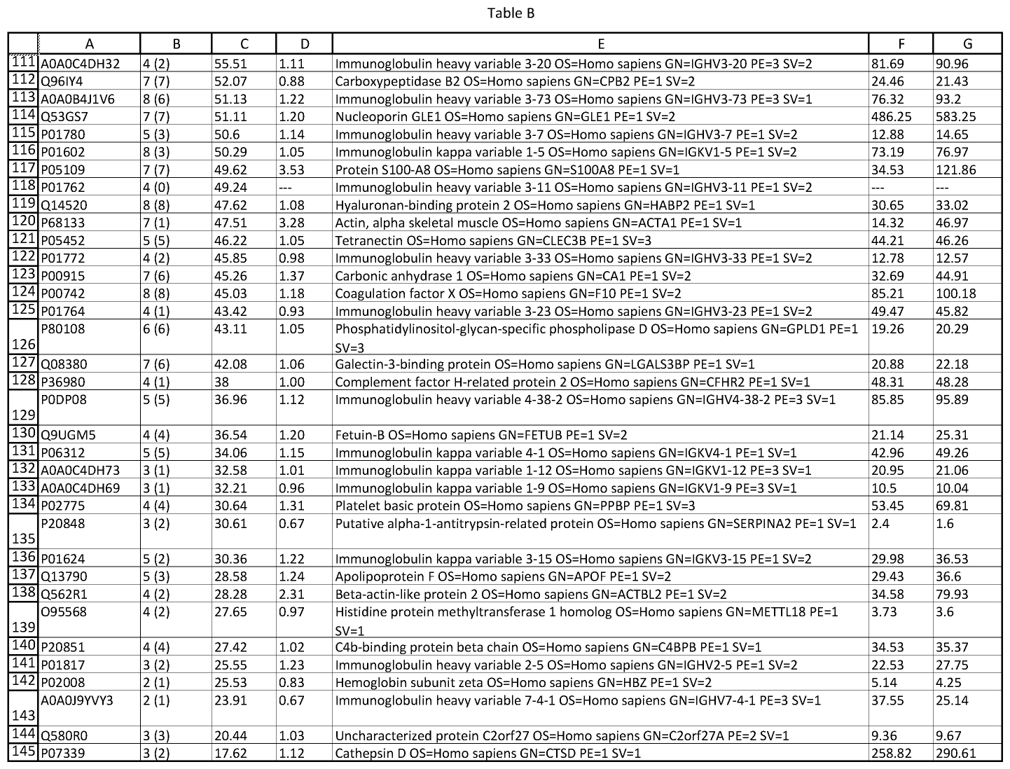

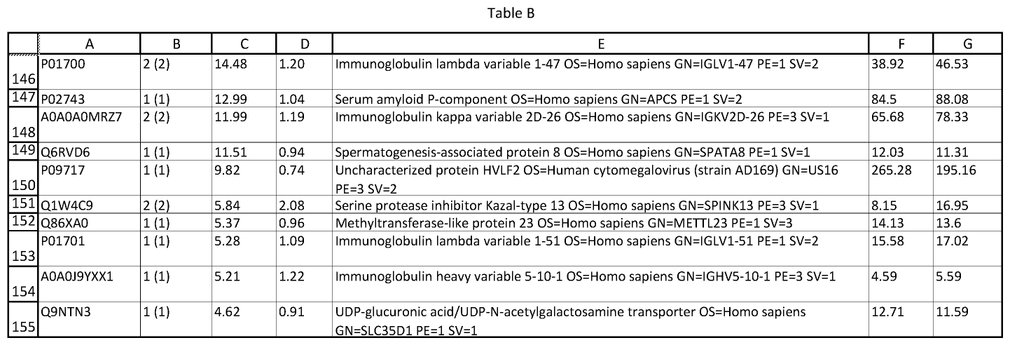

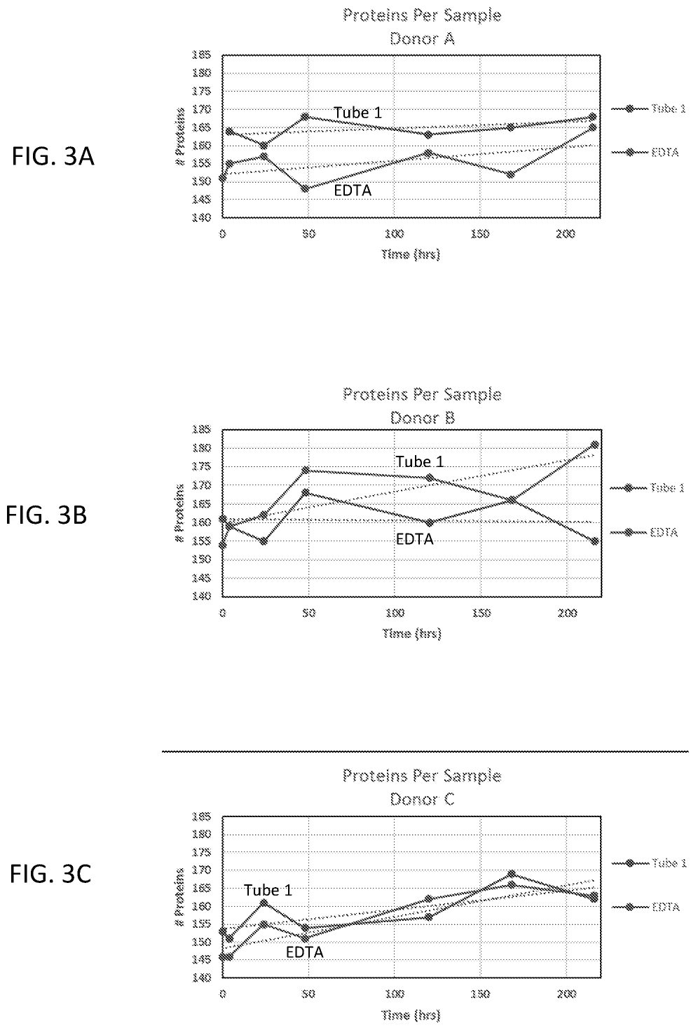

238 proteins were quantified across all samples and were represented by 324 peptides. An average of 160 proteins were quantified in each sample though the number of proteins quantified for each sample (at storage time 0) was greater for samples collected in the CF BCTs compared to E BCTs. Also as shown in FIGS. 3A-3C, the slope of the best fit line was closer to zero for the CF BCTs (Tube 1) compared to that of the E BCTs (EDTA) supporting that the number of proteins quantified in samples collected in CF BCTs was less impacted by storage time compared to samples collected in E BCTs.

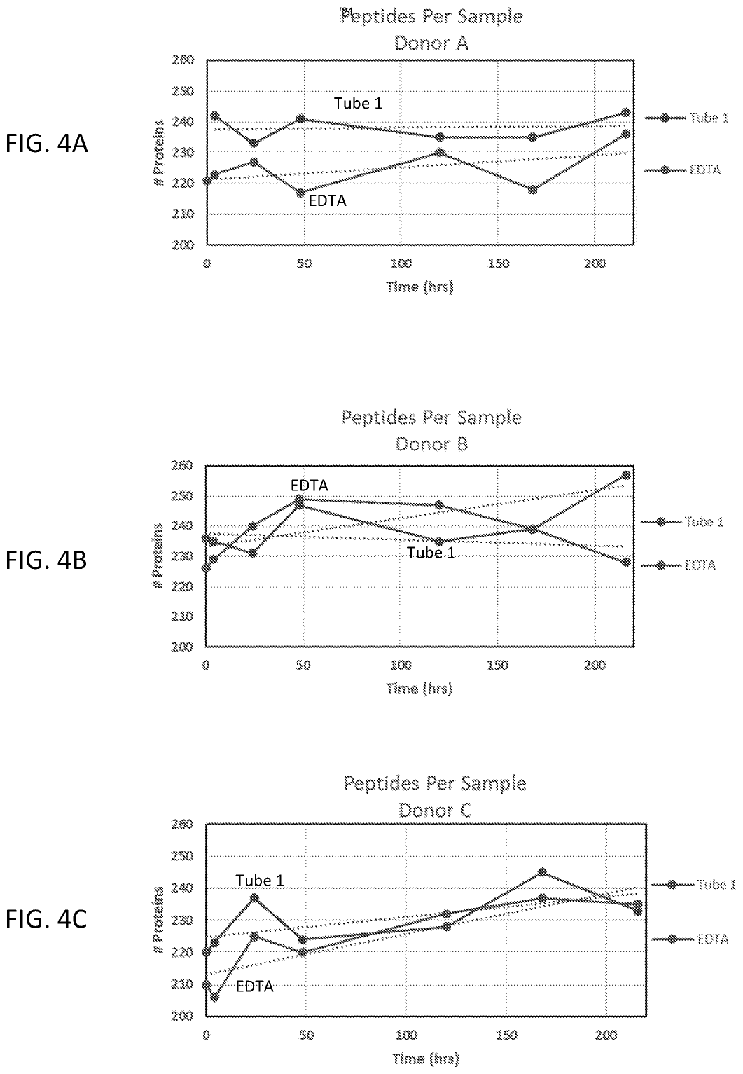

324 peptides were quantified across all samples. An average of 232 peptides were quantified in each sample though the number of peptides quantified for each sample (at storage time 0) was greater for samples collected in the CF BCTs compared to E BCTs. Also as shown in FIGS. 4A-4C, the slope of the best fit line was closer to zero for the CF BCTs (Tube 1) compared to that of the E BCTs (EDTA) supporting that the number of peptides quantified in samples collected in CF BCTs was less impacted by storage time compared to samples collected in E BCTs.

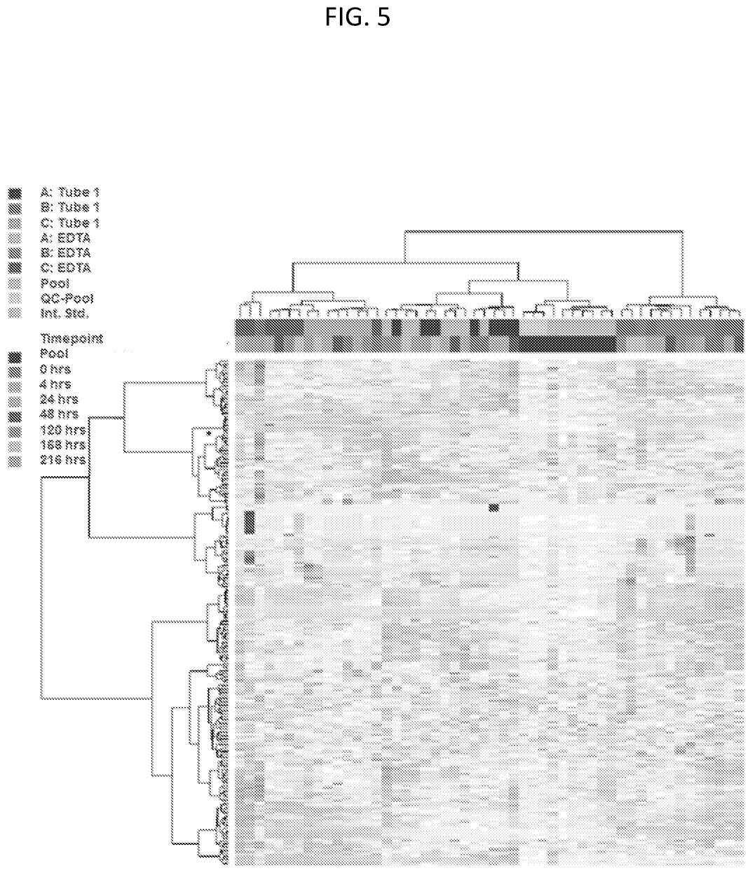

Heirarchal clustering analyses were carried out. A sample dendogram is shown in FIG. 5 and displays strong separation according to donor. Also there is a strong secondary separation according to BCT.

Proteins for further evaluation were selected by examining the top 25 candidate proteins ranked by contribution to component 1 of a Partial Least Squares Discriminant Analysis. Those than demonstrated a difference based upon storage in E BCTs versus CF BCTs were evaluated. Proteins that were differentially abundant between plasma collected in CF BCTs vs E BCTs were also selected for further evaluation. There was significant overlap with the top 25 candidate proteins examined above.

A decreased level in cell lysis and contaminating proteins was demonstrated by examination levels of the proteins listed in Table 4:

TABLE 4

| Protein Name | Cellular Location |
|---|---|
| Profilin-1 | Cytoskeleton |
| Thioredoxin | Nucleus, cytoplasm |
| Rho GDP-dissociation inhibitor 2 | Cytosol |
| Macrophage migration inhibitory factor | Cytoplasm |
| Phosphoglycerate Kinase 1 | Cytoplasm |
| Flavin reductase (NADPH) | Cytoplasm |
| Peroxiredoxin-6 | Lysosome, cytoplasm |
| Glutathione S-transferase omega-1 | Cytosol |
| Heat Shock Cognate 71 IDa protein | Nucleus, Plasma membrane, |
| | cytoplasm |
| Carbonic anhydrase 1 | Cytoplasm |
| Carbonic anhydrase 2 | Cell membrane, cytoplasm |
| Hemoglobin subunit beta | Cytosol |
| Hemoglobin subunit delta | Cytosol |
| Hemoglobin subunit alpha | Cytosol |
| SH3 domain-binding glutamic | Nucleus |
| acid-rich-like protein 3 | |
| Transketolase | Cytosol, Nucleus, Peroxisome |

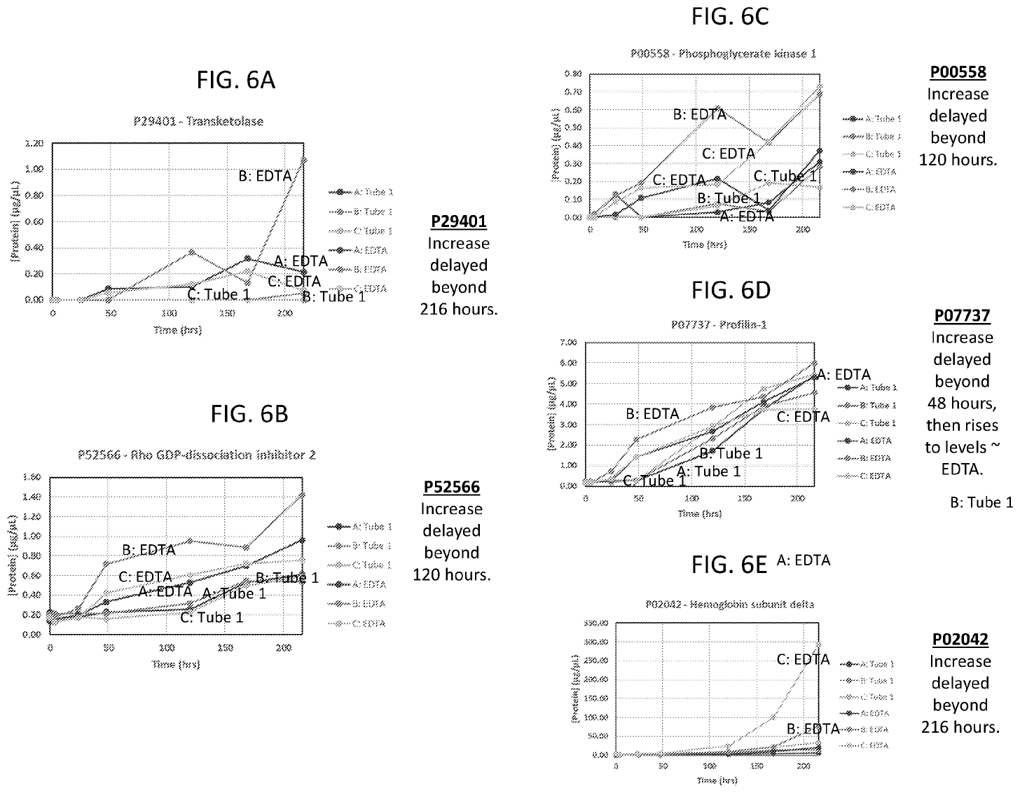

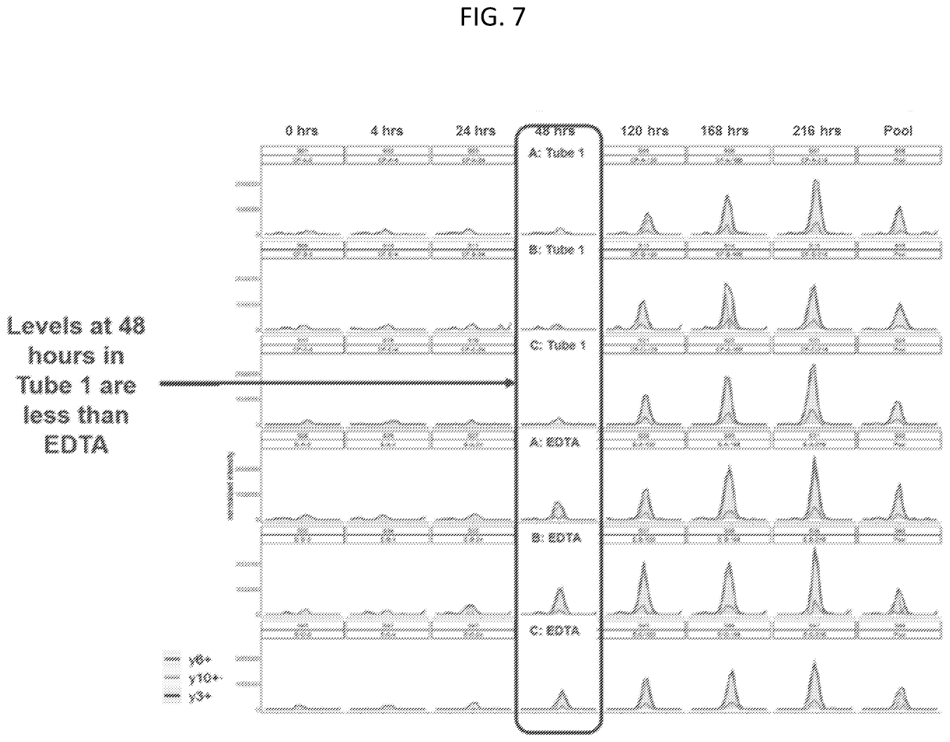

These proteins were at low levels initially and then their levels increased due to storage time. The increase was delayed and/or less significant in samples collected in CF BCTs. As shown in FIG. 6A-6E, the level of protein (transketolase (FIG. 6A); Rho GDP dissociation inhibitor 2 (FIG. 6B); Phosphoglycerate Kinase 1 (FIG. 6C), Profilin (FIG. 6D); Hemoglobin Subunit Delta (FIG. 6E)) was lower over time for each sample collected in a CF BCT vs. samples collected in E BCTs. FIG. 7 provides a chromatographic example, wherein the extracted ion chromatogram for Profilin-1 is shown. At 48 hours, the level of profiling was lower for each sample collected in CF BCTs (Tube A) compared to samples collected in E BCTs.

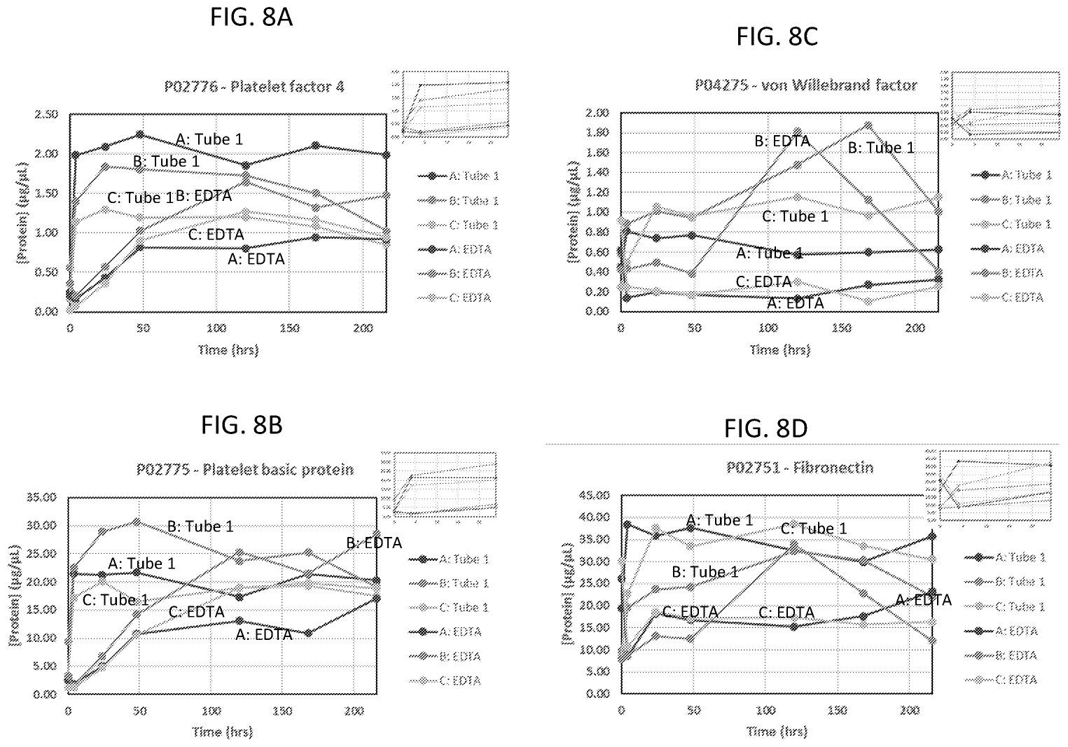

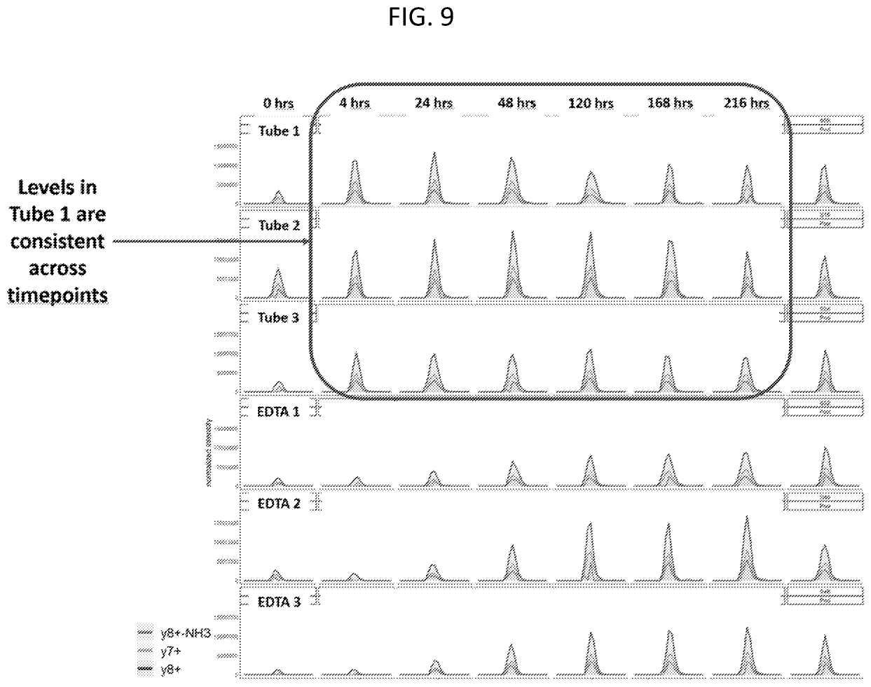

To determine the stability of samples over time, the levels of low abundance and secretory proteins examined. Table 5 lists exemplary proteins analyzed for this purpose. As shown in FIGS. 8A-8D, the levels of low abundance and secretory proteins (Platelet Factor 4 (FIG. 8A); Platelet basic protein (FIG. 8B); von Willebrand factor (FIG. 8C); Fibronectin (FIG. 8D)) were increased for samples collected in CF BCTs (Tube 1) compared to samples collected in E BCTs (EDTA). Also, for the samples collected in CF BCTs the protein level plateaued in less time (within 4 hours) relative to the samples collected in E BCTs. Further for the samples collected in CF BCTs the proteins were less susceptible to degradation (as shown by a decrease in concentration) relative to those samples collected in E BCTs. FIG. 9 provides a chromatographic example wherein the extracted ion chromatogram for Platelet basic protein is shown. Across the 4 hour to 216 hour timeframe, the level of protein was higher for samples collected in CF BCTs (Tube 1) compared to samples collected in E BCTs (EDTA).

TABLE 5

| Protein Name | Cellular Location |
|---|---|
| Platelet basic protein | Extracellular/secreted |
| von Willebrand factor | Extracellular/secreted |
| Thrombospondin-1 | Extracellular/secreted, ER |
| Platelet factor 4 | Extracellular/secreted |
| SPARC | Extracellular/secreted |
| Fibronectin | Extracellular/secreted |
| Coagulation factor IX | Extracellular/secreted |

The data support that the collection and storage of blood in CF BCTs successfully provided samples with improved stability characteristics. Secreted plasma proteins were less susceptible to degradation in CF BCT than EDTA (see P04275/P02751) and/or reached rapid equilibrium (P02776/P02775). Cellular (contaminating) protein release was delayed in samples for 48 to 216 hours (or beyond). These effects enable a consistent sample for extended time periods which would improve analytical results. Furthermore, this stability would enable shipping or transport of the samples to a laboratory for proteomics analysis.

### Example 6

This example describes the materials and methods used in the study of Example 5.

Plasma Samples and Preparation Thereof

A total of 48 human plasma samples were provided by Streck (La Vista, Nebr.) and one control sample was provided by Biognosys AG (Schlieren, Switzerland). Among the samples provided, 42 individual samples were collected in either CF BCTs or E BCTs. Each of the individual samples were collected in three biological replicates corresponding to three individuals. The samples were stored at one of 7 time points ranging from 0 hours to 216 hours.

Chemicals and Reagents

All solvents were HPLC-grade from Sigma-Aldrich and all chemicals where not stated otherwise were obtained from Sigma-Aldrich. No proteolytic enzyme inhibitors were used during any of the steps of this study.

Sample Preparation

Plasma samples were shipped frozen by Streck. One additional plasma for quality control was provided by Biognosys. Samples were denatured using Biognosys' Denature Buffer, reduced using Biognosys' Reduction Solution for 60 min at 60° C. and alkylated using Biognosys' Alkylation Solution for 30 min at room temperature in the dark. Subsequently, digestion to peptides was carried out using 1 μg of trypsin (Promega) per sample overnight at 37° C.

Clean-Up for Mass Spectrometry

Peptides were desalted using a BioPureSPE Midi plate (The Nest Group) according to the manufacturer's instructions and dried down using a SpeedVac system. Peptides were resuspended in 174 LC solvent A (1% acetonitrile, 0.1% formic acid (FA)) and spiked with Biognosys' iRT kit calibration peptides. Peptide concentrations were determined using a UV/VIS Spectrometer (SPECTROstar Nano, BMG Labtech).

Spike-In

Peptides were diluted to 1 μg/μL and spiked with 14 of PQ500™ (Biognosys) containing 1 injection equivalent (IE).

HRM Mass Spectrometry Acquisition

For the DIA LC-MS/MS measurements, 1 μg of peptides containing 1 IE PQ500 per sample were injected to an in-house packed C18 column (Waters CSH C18, 1.7 μm particle size, 130 Å pore size; 75 μm inner diameter, 60 cm length, PicoFrit 10 μm tip, New Objective) on a Thermo Scientific™ Easy nLC 1200 nano-liquid chromatography system connected to a Thermo Scientific Orbitrap Fusion™ Tribrid™ mass spectrometer equipped with a nanoFlex electrospray source. LC solvents were A: water with 0.1% FA; B: 20% water in acetonitrile with 0.1% FA. The nonlinear LC gradient was 1-59% solvent B in 54 min 48 seconds followed by 59-90% B in 10 seconds, 90% B for 7 minutes and 52 seconds, and 90%-1% B in 10 seconds and 1% B for 4 minutes.

HRM Data Analysis

HRM mass spectrometric data were analyzed using Spectronaut™ software (Biognosys) and the PQ500™ assay panel. The false discovery rate on peptide level was set to 1%, data was filtered using row based extraction. The HRM measurements analyzed with Spectronaut were normalized using local regression normalization (Callister et al., J Proteome Res 2006, 5(2), 277-86).

Data Analysis

Absolute protein quantities were calculated from the ratio of SIS peptide to endogenous peptide and adjusted to the injected proportion of original plasma sample. For testing of differential protein abundance, protein concentrations for each protein were analyzed using a two-sample sample Student's t-test. Distance in heat maps was calculated using the “manhattan” method, the clustering using “ward.D” for both axes. Principal component analysis was conducted in R using prcomp and a modified ggbiplot function for plotting, and partial least squares discriminant analysis was performed using mixOMICS package. General plotting was done in R using ggplot2 package.

### Example 7

This example describes an exemplary method of preparing a protein sample for proteomic analysis using one or more mass spectrometry-based proteomic methods, wherein the mass spectrometry of the mass spectrometry-based proteomic methods is a not targeted mass spectrometry.

Blood Sample Collection and Storage: Blood samples were drawn directly into a BCT (CF BCT or E tube) from a donor via venipuncture following the CLSI Approved Standard “Procedures for the Collection of Diagnostic Blood Specimens by Venipuncture”, CLSI Document GP41. Samples were collected in biological replicates corresponding to three individuals. Plasma was isolated at 1, 4, 24, 48, 120, 168 or 216 hours using the double spin protocol described in the Streck Cell-Free DNA BCT IFU. Isolated plasma was stored at −80° C. until processing.

Mass Spectrometry Analysis Sample Preparation: Samples were prepared for mass spectrometry analysis. Briefly, plasma was thawed and then depleted using Pierce™ Top 12 Abundant Protein Depletion Spin Columns (Catalog number 85164, ThermoFisher Scientific, Waltham, Mass.). Samples were desalted and concentrated using Pierce™ 3K molecular weight cut-off (MWCO) filters (Catalog Number 88512, ThermoFisher). Using the Biognosys Sample Preparation Kit, the plasma was subjected to protein denaturation, reduction, and alkylation. The reduction was conducted at 60° C. A digestion step was carried out using Trypsin/Lys-C Mix (Catalog number V5071; Promega, Madison, Wis.). The samples were cleaned up with C18 as described in Example 6 and then spiked with Yeast Alcohol Dehydrogenase (UniProt P00330) for quantitative measurement.

Untargeted Analysis: DIA LC-MS/MS was conducted using samples prepared as described above. See Mass Spectrometry Analysis Sample Preparation. Samples were injected onto a C18 column (Waters T3, 1.0×150 mm, 1.7 μm particle size) on a Waters H-Class UHPLC system connected to a Waters Synapt G2-Si equipped with a ESI source. Data was collected in HDMSe mode (ion mobility enabled). LC solvents were A: water with 0.1% Formic Acid, B: acetonitrile with 0.1% Formic Acid.

Data analysis: Mass spectrometric data was analyzed using Progenesis 01 for Proteomics software (Waters, Milford, Mass.). Positive hits were determined using the Uniprot Human Reference Proteome. The search utilized a trypsin digestion with 2 allowed missed cleavages and a max protein mass of 300 kDa. Fixed modifications were carbamidomethyl cysteine. Variable modifications were oxidation of methionine, n-terminal acetylation, deamidation of glutamine, and deamidation of arginine. Search tolerance parameters were set to automatic for peptide and fragment mass tolerances and the false discovery rate was set to 1%. Ion matching requirements were set to 3 fragments/peptide, 7 fragments/protein, and 1 peptide/protein. Protein quantitation was performed by comparing the top-3 determined peptides to the top-3 peptides arising from yeast alcohol dehydrogenase.

Results

Protein Depletion: Pierce™ Top 2 or Top 12 Abundant Protein Depletion Spin columns used to remove abundant plasma proteins from plasma. Antibodies for the specified proteins are used for protein removal:

| Top 2 and Top 12 | Top 12 Only |
|---|---|
| IgG | α1-Acid Glycoprotein |
| Albumin | α1-Antitrypsin |
| | α2-Macroglubulin |
| | Apolipoprotein A-I |
| | Apolipoprotein A-II |
| | Fibrinogen |
| | Haptoglobin |
| | IgA |
| | IGM |
| | Transferrin |

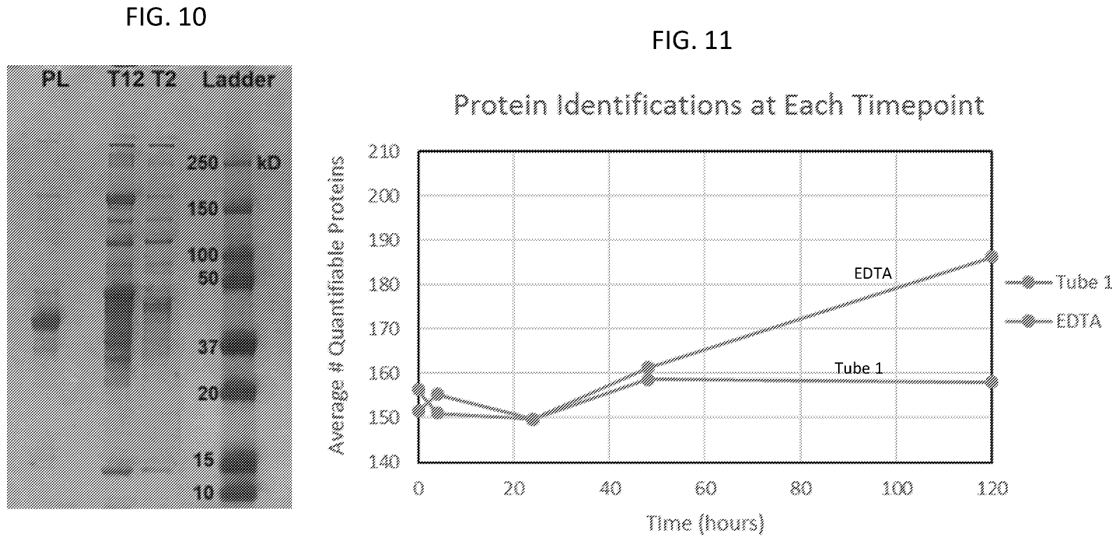

Protein depletion enabled detection of lower abundance proteins in mass spectrometry or gel electrophoresis studies. An exemplary SDS-PAGE of plasma protein following depletion is shown in FIG. 10. The PL lane shows undepleted plasma, the T12 lane shows the Top 12 protein depleted plasm, and the T2 lane shows the Top 2 protein depleted plasma. In the mass spectrometry analysis, the detected serum albumin accounted for 1.65% of all quantified protein in Tube 1 and 1.67% in EDTA. This is reduced from a theoretical concentration of approximately 55% in blood. Tube 1 results are consistent with EDTA and manufacturer's IFU which claim to remove >95%.

Total Proteins: 353 proteins were identified across all samples. Of the 353 proteins, 337 were quantifiable proteins. An average of 165 proteins were identified in each sample. FIG. 11 is a graph of the average number of quantifiable proteins plotted as a function of time for the samples collected with CF BCTs (Tube 1) or with E tubes (EDTA). The number of proteins quantified in samples obtained using CF BCTs at t=0 hours is approximately equivalent to E tubes. The number of proteins quantified in samples collected in CF BCTs is less impacted by storage time than those collected in E tubes.

Individual Protein Evaluations: Proteins for further evaluation were selected by examining protein candidates that were differentially abundant between plasma stored for different periods of time. Those that demonstrated a fold change difference of greater than or equal to 1.5 over the storage period were selected. A decreased level in cell lysis and contaminating proteins is demonstrated by examination levels of the following proteins:

| Protein Name | Cellular Location |
|---|---|
| Protein S100-6 | Nucleus, Plasma membrane |
| Protein S100-A9 | Cytoskeleton, Plasma membrane, |
| | Extracellular |
| Protein S100-A11 | Nucleus |
| Protein S100-A12 | Cytoskeleton, Plasma membrane, |
| | Extracellular |
| Coactosin-like protein | Nucleus, cytoskeleton |
| E3 Ubiquitin-Protein Ligase TRIM7 | Nucleus |
| Hemoglobin subunit alpha | Cytosol |
| Hemoglobin subunit beta | Cytosol |

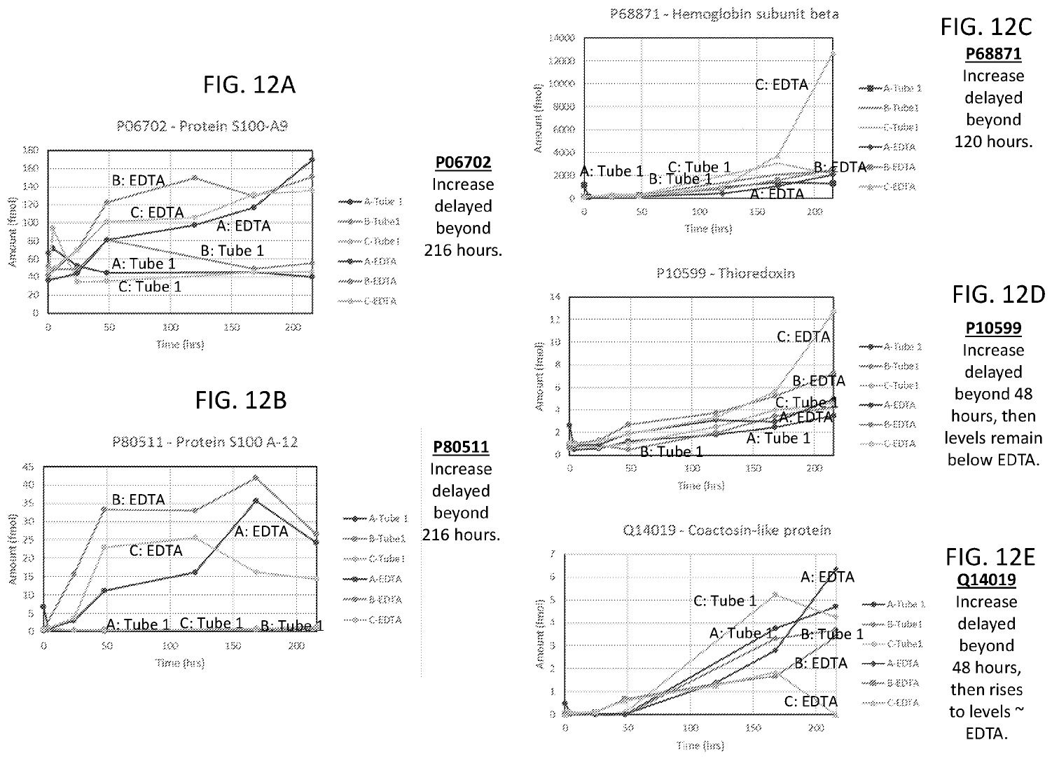

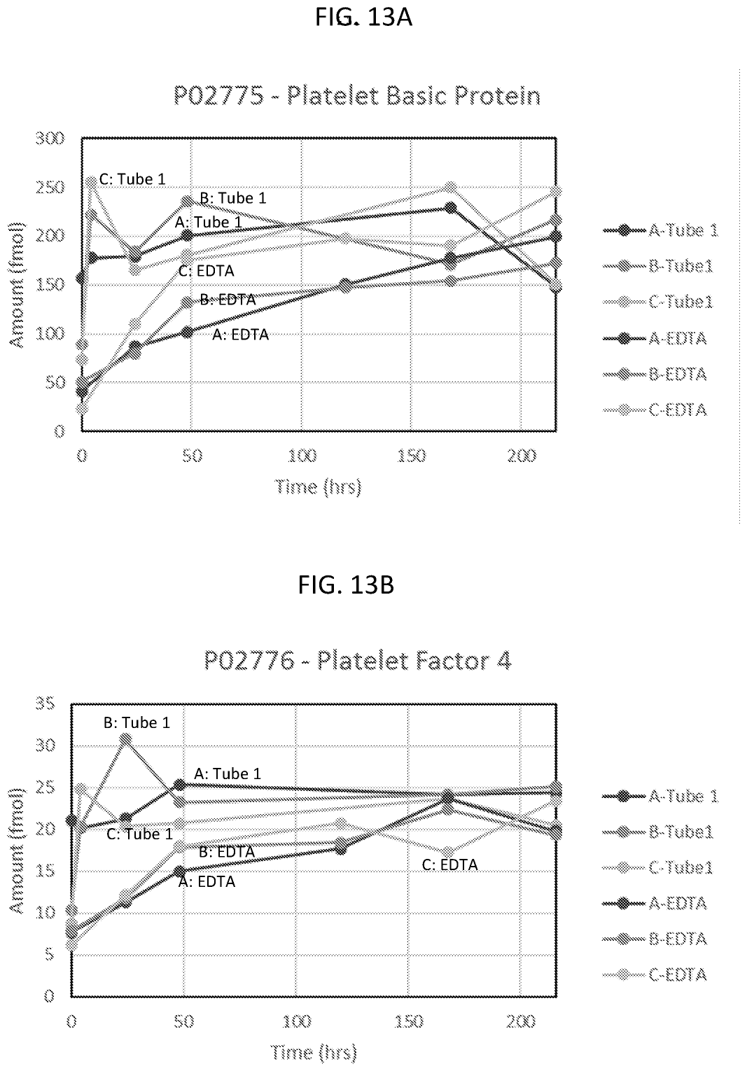

As shown in FIG. 12A-12E, the level of proteins (Protein S100-A9 (FIG. 12A); Protein S100-A12 (FIG. 12B); Hemoglobin subunit beta (FIG. 12C), Thioredoxin (FIG. 13D); Coactosin-like Protein (FIG. 13E)) were low initially and increased due to storage time. These proteins were expected to be found in plasma, but arise from cell lysis. An example is hemoglobin which increases due to lysis of RBCs.

An increased level of low abundance and secretory proteins were demonstrated by examination of the levels of Platelet Basic Protein (FIG. 13A) and Platelet Factor 4 (FIG. 13B). These proteins are at equivalent or higher concentrations in Tube 1 (CF DNA BCT) than EDTA. Proteins in Tube 1 (CF DNA BCT) reached stable level much quicker (i.e. within 4 hours) than in EDTA. Proteins were less susceptible to degradation (i.e. decrease in concentration) in Tube 1 (CF DNA BCT) than EDTA.

This example demonstrated that collecting and storing blood in CF DNA BCT successfully provided samples with improved stability characteristics. Secreted plasma proteins reached rapid equilibrium in CF DNA BCT compared to EDTA as shown by P02776/P02775. Cellular (contaminating) protein release was delated in samples for 48 to 216 hours (or beyond). These effects enable a consistent sample for extended time periods, which provides improved analytical results. This improved sample stability would further enable shipping or transport of the samples to a laboratory for subsequent proteomic analysis.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of preparing a protein sample for proteomic analysis, comprising
a. contacting a blood sample comprising proteins with a protective agent comprising an anticoagulant (AC) and an aldehyde releaser (AR), to obtain a mixture, optionally, wherein the blood sample is added to a blood collection tube (BCT) comprising the protective agent or the blood sample is directly drawn from a subject into a BCT comprising the protective agent, and
b. isolating a fraction comprising proteins from the mixture to yield a protein sample suitable for proteomic analysis,
wherein:
(I) steps (a) and (b) are carried out in the absence of exogenous proteolytic enzyme inhibitors;
(II) the slope of the best fit line of a line graph of the number of proteins in the protein sample yielded from step (b) plotted as a function of storage time is closer to 0 compared to the slope of the best fit line of a line graph of the number of proteins in a control blood sample not contacted with a protective agent;
(III) the number of plasma proteins and/or peptides present in the protein sample following storage for at least 48 hours is within about 10% of the number of plasma proteins and/or peptides present in the protein sample within about 0 hours to about 4 hours of collecting the blood sample from a subject;
(IV) the method further comprises transporting the mixture in a sealed container to a laboratory for proteomic analysis, optionally, wherein the sealed container is a sealed BCT comprising the protective agent; or
(V) any combination thereof.

2. A method of preparing a protein sample for proteomic analysis, comprising
a. contacting a blood sample comprising proteins with a protective agent comprising an anticoagulant (AC) and an aldehyde releaser (AR), to obtain a mixture, optionally, wherein the blood sample is added to a blood collection tube (BCT) comprising the protective agent or the blood sample is directly drawn from a subject into a BCT comprising the protective agent,
b. isolating a cellular fraction comprising a source of cellular proteins from the mixture, and
c. lysing cells of the cellular fraction to yield a protein sample comprising cellular proteins, wherein the protein sample is suitable for proteomic analysis,
wherein:
(I) steps (a) and (b) are carried out in the absence of exogenous proteolytic enzyme inhibitors;
(II) the slope of the best fit line of a line graph of the number of proteins in the protein sample yielded from step (b) plotted as a function of storage time is closer to 0 compared to the slope of the best fit line of a line graph of the number of proteins in a control blood sample not contacted with a protective agent;
(III) the number of plasma proteins and/or peptides present in the protein sample following storage for at least 48 hours is within about 10% of the number of plasma proteins and/or peptides present in the protein sample within about 0 hours to about 4 hours of collecting the blood sample from a subject;
(IV) the method further comprises transporting the mixture in a sealed container to a laboratory for proteomic analysis, optionally, wherein the sealed container is a sealed BCT comprising the protective agent; or
(V) any combination thereof.

3. The method of claim 1 or 2, wherein the protective agent comprises the AR and the AC at a AR to AC ratio of about 1:2 to about 1:6.

4. The method of claim 3, wherein the protective agent comprises the AR and the AC at a AR to AC ratio of about 1:3 to about 1:5.

5. The method of claim 4, wherein the protective agent comprises the AR and the AC at a AR to AC ratio of about 1:4.

6. The method of claim 1 or 2, wherein the AC comprises ethylene diamine tetra acetic acid (EDTA), or a salt thereof, ethylene glycol tetra acetic acid (EGTA) or a salt thereof, hirudin, heparin, citric acid or a salt thereof, oxalic acid or a salt thereof, acid citrate dextrose (ACD), citrate, citrate-theophylline-adenosine-dipuridamole (CTAD), citrate-pyridoxalphosphate-tris, heparin-1,3-hydroxy-ethyl-theophylline, polyanethol sultanate, sodium fluoride, sodium heparin, thrombin and PPACK (D-phenylalanyl-L-prolyl-L-arginine chloromethyl ketone), or a combination thereof.

7. The method claim 6, wherein the AC comprises EDTA, optionally, wherein EDTA is the only AC present in the protective agent.

8. The method of claim 6 or 7, wherein the protective agent comprises about 50 g/l to about 100 g/l AC.

9. The method of any one of the preceding claims, wherein the AR is diazolidinyl urea, imidazolidinyl urea, 1,3,5-tris(hydroxyethyl)-s-triazine, oxazolidines, 1,3-bis(hydroxymethyl)-5,5-dimethylimidazolidine-2,4-dione, quaternium-15, DMDM hydantoin, 2-bromo-2-nitropropane-1,3-diol, 5-bromo-5-nitro-1,3-dioxane, tris(hydroxymethyl) nitromethane, hydroxymethylglycinate, polyquaternium, or a combination thereof.

10. The method of claim 9, wherein the AR comprises imidazolidinyl urea, optionally, wherein imidazolidinyl urea is the only AR in the protective agent.

11. The method of claim 9 or 10, wherein the protective agent comprises about 0.1 g/ml to about 3 g/ml AR.

12. The method of any one of preceding claims, wherein the protective agent further comprises an amine.

13. The method of claim 12, wherein the amine is tryptophan, tyrosine, phenylalanine, glycine, ornithine and S-adenosylmethionine, aspartate, glutamine, alanine, arginine, cysteine, glutamic acid, glutamine, histidine, leucine, lysine, proline, serine, threonine, or a combination thereof.

14. The method of claim 13, wherein the amine is glycine, optionally, wherein glycine is the only amine in the protective agent.

15. The method of claim 13 or 14, wherein the protective agent comprises about 20 g/l to about 60 g/l amine.

16. The method of any one of claims 12 to 15, wherein the amount of amine relative to the amount of is about 1 part by weight amine to about 10 parts by weight AR.

17. The method of any one of the preceding claims, wherein the protective agent comprises not more than about 50,000 ppm formaldehyde.

18. The method of any one of the preceding claims, wherein the protective agent comprises imidazolidinyl urea, EDTA, and glycine.

19. The method of any one of the previous claims, wherein the protective agent consists essentially of (i) about 300 g/l to about 700 g/l imidazolidinyl urea; (ii) about 20 g/l to about 60 g/l glycine; and (iii) about 60 g/l to about 100 g/l EDTA.

20. The method of any one of the preceding claims, comprising isolating a plasma fraction from the blood sample to yield a protein sample suitable for proteomic analysis.

21. The method of any one of claims 1 to 19, wherein the fraction is a cellular fraction isolated from the mixture.

22. The method of claim 21, wherein the cellular fraction consists essentially of rare blood cells, optionally, wherein the rare blood cells are circulating tumor cells (CTCs).

23. The method of claim 21 or 22, wherein rare blood cells of the blood sample are separated from other cells in the blood sample, optionally, wherein rare blood cells of the blood sample are separated from red blood cells, white blood cells, platelets, or a combination thereof.

24. The method of any one of claims 21 to 23, wherein rare blood cells of the blood sample are separated from plasma proteins.

25. The method of any one of claims 21 to 25, comprising lysing cells of the cellular fraction to obtain a protein sample suitable for proteomic analysis.

26. The method of any one of the preceding claims, wherein the mixture had been stored for at least 48 hours prior to step (b), for at least 48 hours but less than 7 days prior to step (b), or for at least 48 hours but less than 14 days prior to step (b).

27. The method of claim 26, wherein the mixture had been stored at a temperature greater than or about 4° C., optionally, at a temperature of about 20° to about 25° C.

28. The method of any one of the preceding claims, comprising storing the mixture in the BCT for at least 48 hours prior to step (b), for at least 48 hours but less than 7 days prior to step (b), or for at least 48 hours but less than 14 days prior to step (b).

29. The method of claim 28, comprising storing the mixture in the BCT at a temperature greater than or about 4° C., optionally, at a temperature of about 20° C. to about 25° C.

30. The method of any one of the preceding claims, wherein the isolating step comprises:
a. depleting one or more proteins from the sample;
b. adding a digestion enzyme, a reducing agent, an alkylating agent, to the sample;
c. identifying proteins present in the sample;
d. quantitating total and individual protein concentration of the sample or an aliquot thereof;
e. labeling proteins with a tag; or
f. a combination thereof.

31. The method of claim 30, comprising depleting immunoglobulins, albumin, or both from the sample.

32. The method of claim 30 or 31, wherein (i) the digestion enzyme is trypsin, (ii) the reducing agent comprises urea or dithiothreitol (DTT) or both, (iii) the alkylating agent comprises iodoacetamide (IAA), or (iv) a combination thereof.

33. The method of any one of the preceding claims, wherein the protein sample suitable for proteomic analysis is characterized by:
(a) a decreased level in cell lysis;
(b) a decreased level in contaminant proteins;
(c) an increased level of low-abundance plasma proteins;
(d) an increased level of unique peptides identified per protein
(e) an increased level of unique proteins identified as determined by LC-MS/MS, optionally, wherein the unique proteins are secretory proteins; or
(f) a combination thereof;
compared to the amount of a control protein sample obtained from an isolated fraction of a blood sample collected in a blood collection tube without a protective agent (e.g., comprising only EDTA),
following storage for at least 48 hours, for at least 48 hours but less than 7 days, or for at least 48 hours but less than 14 days, at a temperature greater than or about 4° C., prior to the isolating step, optionally, at a temperature of about 20° C. to about 25° C.,

34. The method of any one of the preceding claims, wherein the protein sample comprises greater than about 70% intact proteins present in a freshly isolated blood sample.

35. The method of any one of the preceding claims, wherein the protein sample comprises less than about 40% contaminant protein products present in a blood sampled stored without the protective agent for more than 48 hours at a temperature about 4° C.

36. The method of any one of the preceding claims, further comprising analyzing the proteins in the protein sample using one or more mass spectrometry-based proteomic methods.

37. The method of claim 36, wherein the mass spectrometry of the mass spectrometry-based proteomic methods comprises a targeted mass spectrometry.

38. The method of claim 37, wherein the mass spectrometry experiment utilizes parallel reaction monitoring (PRM), selected reaction monitoring (SRM), selected ion monitoring (SIM), or multiple reaction monitoring (MRM).

39. The method of claim 37, wherein the mass spectrometry of the mass spectrometry-based proteomic methods is a not targeted mass spectrometry.

40. The method of claim 39, wherein the mass spectrometry experiment utilizes data-dependent acquisition (DDA), data independent acquisition (DIA), or labeled quantitation (e.g. tandem mass tag (TMT)) mass spectrometry.

41. A method of preparing a protein sample for proteomic analysis, comprising
a. adding a blood sample comprising proteins into a blood collection tube (BCT) comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA;
b. optionally, storing the blood sample in the BCT for at least about 48 hours at about 20° C. to about 25° C.;
c. isolating a fraction comprising proteins, yielding a protein sample suitable for proteomic analysis and
d. analyzing the protein sample via one or more mass spectrometry-based proteomic methods,
wherein steps of the method are carried out without the use of any exogenous proteolytic enzyme inhibitors.

42. A method of preparing a protein sample for proteomic analysis, comprising
a. adding a blood sample comprising proteins into a blood collection tube (BCT) comprising a protective agent consisting essentially of (i) about 300 g/l to about 700 g/l imidazolidinyl urea; (ii) about 20 g/l to about 60 g/l glycine; and (iii) about 60 g/l to about 100 g/l EDTA;
b. optionally, storing the blood sample in the BCT for at least about 48 hours at about 20° C. to about 25° C.;
c. isolating a cellular fraction comprising a source of cellular proteins from the mixture;
d. lysing cells of the cellular fraction to yield a protein sample comprising cellular proteins, wherein the protein sample is suitable for proteomic analysis and
e analyzing the protein sample via one or more mass spectrometry-based proteomic methods;
wherein steps of the method are carried out without the use of any exogenous proteolytic enzyme inhibitors.
